(19) United States
(12) Patent Application Publication
(10) Pub. No.: US 2022/0113301 A1
(43) Pub. Date: Apr. 14, 2022

(54) CELL ANALYZER, METHOD FOR CLASSIFYING WHITE BLOOD CELL BASED ON IMPEDANCE METHOD, AND COMPUTER-READABLE STORAGE MEDIUM

(71) Applicant: Shenzhen Mindray Animal Medical Technology Co., Ltd, Shenzhen (CN)

(72) Inventor: Fangang KONG, Shenzhen (CN)

(21) Appl. No.: 17/555,281

(22) Filed: Dec. 17, 2021

Related U.S. Application Data
(63) Continuation of application No. PCT/CN2019/091891, filed on Jun. 19, 2019.

(51) Int. Cl. G01N 33/50; B01L 3/00; G01N 1/44; G01N 15/10; G01N 1/14

(52) U.S. Cl. CPC G01N 33/5094; G01N 2015/008; G01N 1/44; G01N 15/10; G01N 1/14; B01L 2200/025; B01L 2200/026; B01L 2200/10; B01L 2200/16; B01L 2300/0663; B01L 2300/0838; B01L 2300/14; B01L 2300/1805; B01L 2400/0487; B01L 3/502

(57) ABSTRACT

Disclosed are a cell analyzer and a method for classifying white blood cells based on an impedance method. The method includes: adding a sample to be analyzed to a white blood cell counting chamber; adding a hemolytic agent to the white blood cell counting chamber at least once; controlling the temperature of the liquid in the white blood cell counting pool to be within a predetermined range; and analyzing the liquid in the white blood cell counting chamber to classify white blood cells into at least four classifications and counting.

### CROSS-REFERENCE TO RELATED APPLICATIONS

This application is a continuation of International Application No. PCT/CN2019/091891, filed Jun. 19, 2019, the content of which is incorporated herein by reference.

### TECHNICAL FIELD

The present disclosure relates to a cell analyzer and a method for classifying white blood cell based on an impedance method.

### BACKGROUND

Human white blood cells are classified into five types: lymphocytes, monocytes, neutrophils, eosinophils and basophils. Clinicians can diagnose a patient's condition according to count and percentage of each type of white blood cells and combined with the patient's clinical symptoms.

White blood cells of mammals, such as dogs and cats, are morphologically similar to those of human, and are also classified into five types: lymphocytes, monocytes, neutrophils, eosinophils and basophils. Similarly, clinicians can also diagnose an animal's condition according to count and percentage of each type of white blood cells and combined with the animal's clinical symptoms, and provide a basis for the animal's diagnosis and the evaluation of therapeutic effect.

Blood cell analyzers for testing blood samples available in the market, for example, taking blood cell analyzers for testing blood samples of animals as an example, can generally have two types as follows. One type is a blood cell analyzer which realizes five-classification of white blood cells by laser flow cytometry, and such type of blood cell analyzer has high classification accuracy, but has high complete machine cost and thus is not conducive to popularization in middle and small pet hospitals. The other type is a blood cell analyzer based on an impedance method, and such type of blood cell analyzer can realize three-classification or four-classification of white blood cells, and is also cost-controllable and is therefore widely used in some middle- and low-grade occasions, such as middle and low-grade hospitals for human or middle and small hospitals for pets, etc. A white blood cell classification based on an impedance method will be described in detail below.

Referring to FIG. 1, in the white blood cell classification realized based on the impedance method, a blood sample is firstly treated with a hemolytic agent for lysing red blood cells in the blood sample to reduce the influence of red blood cells on the classification and counting of white blood cells. Moreover, various types of white blood cells are different in cell volume, and under the action of the hemolytic agent, the various types of white blood cells will shrink in different degrees, such that the difference in volume size among the various types of white blood cells will be further magnified. Then, under the action of a negative pressure, blood cells pass through a detection orifice in a white blood cell counting chamber one by one, wherein a constant-current source is applied across the detection orifice. When a cell passes through the detection orifice, a corresponding pulse will be generated. The larger the cell volume is, the greater the resistance increases when the cell passes through the detection orifice, and thus the larger the pulse is generated. i.e., the amplitude of the pulse is directly proportional to the cell volume, and the frequency of the pulse is directly proportional to the number of cells.

In theory, the above-mentioned method can be used for classifying white blood cells into five types, i.e., respectively obtaining the proportion and count of each of the five types of white blood cells including lymphocytes, monocytes, neutrophils, eosinophils and basophils. However, in practice, it is generally only possible to realize three-classification of white blood cells, i.e., the classification and counting of lymphocytes, monocytes and granulocytes, with eosinophils, basophils and neutrophils being impossible to be further distinguished among the granulocytes. In some solutions, four-classification of white blood cells can be realized through improvement of the composition of hemolytic agents, i.e., it is also possible to differentiate eosinophils from granulocytes, but the accuracy is still not satisfactory.

During the study of those skilled in the art on the white blood cell classification based on an impedance method, they currently focus on improving hemolytic agents, i.e., it is desired to obtain a hemolytic agent by which various types of white blood cells are more obvious in volume difference and more differentiable, thereby achieving accurate four-classification or even five-classification of white blood cells.

### SUMMARY

The disclosure provides a cell analyzer and a method for classifying white blood cells based on an impedance method, which will be described in detail below.

According to a first aspect, in an embodiment, a method for classifying white blood cells based on an impedance method is provided, comprising:

adding a diluent to a white blood cell counting chamber;

adding a sample to be analyzed to the white blood cell counting chamber;

adding a hemolytic agent to the white blood cell counting chamber at least once;

controlling a temperature of the liquid in the white blood cell counting chamber to be within a preset temperature range; and

testing the liquid in the white blood cell counting chamber to perform at least four-classification and counting of white blood cells.

In an embodiment, the step of controlling a temperature of the liquid in the white blood cell counting chamber to be within a preset temperature range comprises: heating the diluent to a certain temperature, and then adding the diluent to the white blood cell counting chamber so as to control the temperature of the liquid in the white blood cell counting chamber to be within the preset temperature range.

In an embodiment, the step of controlling a temperature of the liquid in the white blood cell counting chamber within a preset temperature range comprises: heating the liquid in the white blood cell counting chamber so as to control the temperature of the liquid in the white blood cell counting chamber to be within the preset temperature range.

In an embodiment, the method comprises: adding the hemolytic agent to the white blood cell counting chamber only once such that an amount of red blood cell fragments in the sample is less than a preset threshold; testing the liquid in the white blood cell counting chamber once to obtain a white blood cell histogram; and performing the at least four-classification and counting of white blood cells according to the white blood cell histogram from said testing.

In an embodiment, the method comprises: adding a first hemolytic agent to a cell counting chamber; testing the liquid in the white blood cell counting chamber once to obtain a first white blood cell histogram; adding a second hemolytic agent to the white cell counting chamber such that an amount of red blood cell fragments in the sample is less than a preset threshold; testing the liquid in the white blood cell counting chamber once to obtain a second white blood cell histogram; and performing the at least four-classification and counting of white blood cells according to the first white blood cell histogram and the second white blood cell histogram.

In an embodiment, the first hemolytic agent and the second hemolytic agent are a same hemolytic agent.

In an embodiment, the method comprises: adding the hemolytic agent to the white blood cell counting chamber only once; testing the liquid in the white blood cell counting chamber once to obtain a first white blood cell histogram; waiting for a preset period of time to enable the hemolytic agent to continue to act on the sample such that an amount of red blood cell fragments in the sample is less than a preset threshold; testing the liquid in the white blood cell counting chamber once to obtain a second white blood cell histogram; and performing the at least four-classification and counting of white blood cells according to the first white blood cell histogram and the second white blood cell histogram.

According to a second aspect, in an embodiment, a method for classifying white blood cells based on an impedance method is provided, comprising:

adding a diluent to a white blood cell counting chamber;

aspirating a sample to be analyzed by means of a sampling needle assembly, and discharging a portion of the sample to the white blood cell counting chamber;

adding a first hemolytic agent to the white blood cell counting chamber;

testing the liquid in the white blood cell counting chamber once to obtain a first white blood cell histogram;

draining and cleaning the white blood cell counting chamber;

adding a diluent to the white blood cell counting chamber;

discharging at least a portion of the remaining sample to the white blood cell counting chamber by means of the sampling needle assembly;

adding a second hemolytic agent to the white blood cell counting chamber;

testing the liquid in the white blood cell counting chamber once to obtain a second white blood cell histogram; and

performing at least four-classification and counting of white blood cells according to the first white blood cell histogram and the second white blood cell histogram, wherein an amount of red blood cell fragments in the second white blood cell histogram is less than the preset threshold, wherein a temperature of the liquid in the white blood cell counting chamber is controlled to be within a preset temperature range before each testing.

According to a third aspect, in an embodiment a cell analyzer is provided, comprising:

a white blood cell counting chamber comprising an orifice;

a sampling needle assembly for discharging a sample to be analyzed into the white blood cell counting chamber;

a diluent delivery component for delivering a diluent to the white blood cell counting chamber;

a hemolytic agent delivery component for delivering a hemolytic agent to the white blood cell counting chamber;

a pressure source component for providing pressure to enable the liquid in the white blood cell counting chamber to pass through the orifice;

a resistive detector for testing the liquid passing through the orifice;

a heating component for controlling a temperature of the liquid in the white blood cell counting chamber; and

a controller and a processor; wherein

the controller controls the diluent delivery component to deliver the diluent to the white blood cell counting chamber;

the controller controls the sampling needle assembly to add the sample to be analyzed to the white blood cell counting chamber;

the controller controls the hemolytic agent delivery component to add the hemolytic agent at least once to the white blood cell counting chamber;

the controller controls the heating component to control the temperature of the liquid in the white blood cell counting chamber to be within a preset temperature range;

the controller controls the pressure source component to provide pressure to enable the liquid in the white blood cell counting chamber to pass through the orifice, and controls the resistive detector to test the liquid passing through the orifice; and

the processor performs at least four-classification and counting of white blood cells according to data output by the resistive detector.

According to a fourth aspect, in an embodiment a cell analyzer is provided, comprising:

a white blood cell counting chamber comprising an orifice;

a sampling needle assembly for discharging a sample to be analyzed into the white blood cell counting chamber;

a diluent delivery component for delivering diluent to the white blood cell counting chamber;

a hemolytic agent delivery component for delivering a hemolytic agent to the white blood cell counting chamber;

a cleaning component for cleaning the white blood cell counting chamber;

a heating component for controlling a temperature of the liquid in the white blood cell counting chamber; and

a pressure source component for providing pressure to enable the liquid in the white blood cell counting chamber to pass through the orifice;

a resistive detector for testing the liquid passing through the orifice;

a controller and a processor; wherein

the controller controls the diluent delivery component to deliver the diluent to the white blood cell counting chamber;

the controller controls the sampling needle assembly to aspirate a sample to be analyzed, and discharge a portion of the sample to the white blood cell counting chamber;

the controller controls the hemolytic agent delivery component to add a first hemolytic agent to the white blood cell counting chamber;

the controller controls the pressure source component to provide pressure to enable the liquid in the white blood cell counting chamber to pass through the orifice, and controls the resistive detector to test the liquid passing through the orifice, and the processor obtains a first white blood cell histogram according to data output by the resistive detector in said testing;

the controller controls discharge of the liquid from the white blood cell counting chamber, and controls the cleaning component to clean the white blood cell counting chamber;

the controller controls the diluent delivery component to deliver the diluent to the white blood cell counting chamber;

the controller controls the sampling needle assembly to discharge at least a portion of the remaining sample to the white blood cell counting chamber;

the controller controls the hemolytic agent delivery component to add a second hemolytic agent to the white blood cell counting chamber;

the controller controls the pressure source component to provide pressure to enable the liquid in the white blood cell counting chamber to pass through the orifice, and controls the resistive detector to test the liquid passing through the orifice, and the processor obtains a second white blood cell histogram according to data output by the resistive detector in said testing;

the processor performs at least four-classification and counting of white blood cells according to the first white blood cell histogram and the second white blood cell histogram, wherein an amount of red blood cell fragments in the second white blood cell histogram is less than a preset threshold; and

wherein the controller controls the heating component to control the temperature of the liquid in the white blood cell counting chamber to be within a preset temperature range before each testing.

According to a fifth aspect, in an embodiment a cell analyzer is provided, comprising:

a reaction chamber;

a sampling needle assembly for discharging a sample to be analyzed into the reaction chamber;

a diluent delivery component for delivering a diluent to the reaction chamber;

a hemolytic agent delivery component for delivering a hemolytic agent to the reaction chamber;

a flow chamber for cells in the sample to be analyzed to pass through one by one;

a resistive detector for testing the cells passing through the flow chamber;

a heating component for controlling a temperature of the liquid in the reaction chamber; and

a controller and a processor; wherein

the controller controls the diluent delivery component to deliver the diluent to the reaction chamber;

the controller controls the sampling needle assembly to add the sample to be analyzed to the reaction chamber;

the controller controls the hemolytic agent delivery component to add the hemolytic agent to the reaction chamber at least once;

the controller controls the heating component to control the temperature of the liquid in the reaction chamber to be within the preset temperature range;

the controller controls the cells in the liquid in the reaction chamber to pass through the flow chamber one by one, and controls the resistive detector to test the cells passing through the flow chamber; and

the processor performs at least four-classification and counting of white blood cells according to data output by the resistive detector.

According to a sixth aspect, in an embodiment a cell analyzer is provided, comprising:

a reaction chamber;

a sampling needle assembly for discharging a sample to be analyzed into the reaction chamber;

a diluent delivery component for delivering a diluent to the reaction chamber;

a hemolytic agent delivery component for delivering a hemolytic agent to the reaction chamber;

a flow chamber for cells in the sample to be analyzed to pass through one by one;

a cleaning component for cleaning the reaction chamber;

a heating component for controlling a temperature of the liquid in the reaction chamber;

a resistive detector for testing the cells passing through the flow chamber; and

a controller and a processor; wherein

the controller controls the diluent delivery component to deliver the diluent to the reaction chamber;

the controller controls the sampling needle assembly to aspirate a sample to be analyzed, and discharge a portion of the sample to the reaction chamber;

the controller controls the hemolytic agent delivery component to add a first hemolytic agent to the reaction chamber;

the controller controls the liquid in the reaction chamber to pass through the flow chamber, and controls the resistive detector to test the cells passing through the flow chamber, and the processor obtains a first white blood cell histogram according to data output by the resistive detector in said testing;

the controller controls discharge of the liquid from the reaction chamber, and controls the cleaning component to clean the reaction chamber;

the controller controls the diluent delivery component to deliver the diluent to the reaction chamber;

the controller controls the sampling needle assembly to discharge at least a portion of the remaining sample to the reaction chamber;

the controller controls the hemolytic agent delivery component to add a second hemolytic agent to the reaction chamber;

the controller controls the liquid in the reaction chamber to pass through the flow chamber, and controls the resistive detector to test the cells passing through the flow chamber, and the processor obtains a second white blood cell histogram according to data output by the resistive detector in said testing; and

the processor performs at least four-classification and counting of white blood cells according to the first white blood cell histogram and the second white blood cell histogram, wherein an amount of red blood cell fragments in the second white blood cell histogram is less than a preset threshold; and

wherein the controller controls the heating component to control the temperature of the liquid in the reaction chamber to be within the preset temperature range before each testing.

According to a seventh aspect, in an embodiment, a computer-readable storage medium is provided, comprising a program, which is executable by a processor to implement the method in any one of the above embodiments.

### BRIEF DESCRIPTION OF THE DRAWINGS

FIG. 1 is a structural schematic diagram of a system for realizing white blood cell classification based on an impedance method;

FIG. 2 is a structural schematic diagram of a cell analyzer according to an embodiment;

FIG. 3 is a structural schematic diagram of a cell analyzer according to another embodiment;

FIG. 4 is a structural schematic diagram of a heating component according to an embodiment;

FIG. 5 is a structural schematic diagram of a heating component according to another embodiment;

FIG. 6 is a structural schematic diagram of a helical pipeline provided in a hemolytic agent delivery component according to an embodiment;

FIG. 7 is a structural schematic diagram of a cell analyzer according to still another embodiment;

FIG. 8 is a structural schematic diagram of a cell analyzer according to yet another embodiment;

FIG. 9 is a flow diagram of a method for classifying white blood cells based on an impedance method according to an embodiment;

FIG. 10 is a flow diagram of a method for classifying white blood cells based on an impedance method according to another embodiment;

FIG. 11 is a white blood cell histogram obtained from testing on a dog's blood sample in an example;

FIG. 12A is a white blood cell histogram obtained from testing on a dog's blood sample after a first treatment in an example; FIG. 12B is a white blood cell histogram obtained from testing on the dog's blood sample after a second treatment in an example;

FIG. 13 is a white blood cell histogram obtained from testing on a cat's blood sample in an example;

FIG. 14A is a white blood cell histogram obtained from testing on a cat's blood sample after a first treatment in an example; and FIG. 14B is a white blood cell histogram obtained from testing on the cat's blood sample after a second treatment in an example.

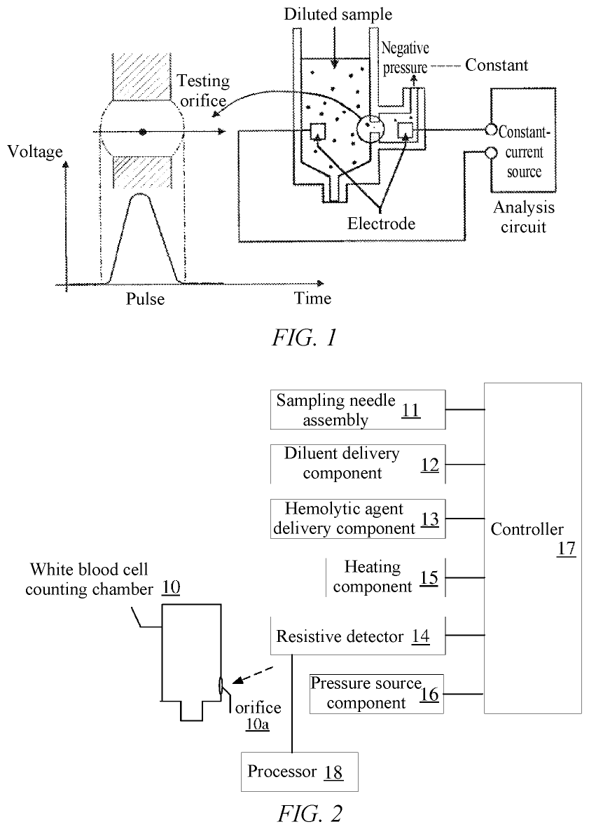

### DETAILED DESCRIPTION

The disclosure will be further described in detail below through specific implementations in conjunction with the accompanying drawings. Associated similar element reference numerals are used for similar elements in different implementations. In the following implementations, many details are described such that the present application can be better understood. However, it may be effortlessly appreciated by a person skilled in the art that some of the features may be omitted, or may be substituted by other elements, materials, and methods in different cases. In certain cases, some operations involved in the present application are not displayed or described in the specification, which is to prevent a core part of the present application from being obscured by too much description. Moreover, for a person skilled in the art, the detailed description of the involved operations is not necessary, and the involved operations can be thoroughly understood according to the description in the specification and the general technical knowledge in the art.

In addition, the characteristics, operations, or features described in the specification can be combined in any appropriate manner to form various implementations. Moreover, the steps or actions in the method description may also be exchanged or adjusted in order in such a way that is apparent to a person skilled in the art. Therefore, the various orders in the specification and the accompanying drawings are merely for the purpose of clear description of a certain embodiment and are not meant to be a necessary order unless otherwise stated that a certain order must be followed.

The serial numbers themselves for the components herein, for example, “first” and “second”, are merely used to distinguish the described objects, and do not have any sequential or technical meaning. Moreover, as used in the present application, “connection” or “coupling”, unless otherwise specified, comprises both direct and indirect connections (couplings).

When those skilled in the art study white blood cell classification based on an impedance method, they focus on the technical route of improving hemolytic agents, and the main task for this technical route of improving hemolytic agents is how to clearly distinguish cell populations in white blood cells under the action of the hemolytic agent such that the various cell populations have clearer boundaries and stronger clustering. In addition, in this technical route, it is often underlined that the hemolytic agents can adapt to a wide range of temperature, and has no special requirements for the temperature during treatment and testing of blood samples.

In the process of studying the white blood cell classification based on the impedance method, the inventors of the present application also follow the existing technical teachings and studies how to improve the hemolytic agents and increase the temperature adaptation range of the hemolytic agents. While advancing on this technical route, when specifically studying how to increase the temperature adaptation range of the hemolytic agents, it occurs to the inventors that expected results may be achieved by using influence of temperature on the hemolytic agents instead of eliminating and reducing the adverse side effect of temperature on the hemolytic agents. The inventors finally propose another technical route of realizing the white blood cell classification by means of an impedance method, in which through temperature control during treatment and testing of blood samples, various types of white blood cells are more obvious in volume difference and more differentiable under the action of the hemolytic agents, so as to finally achieve relatively accurate four-classification or even five-classification of white blood cells.

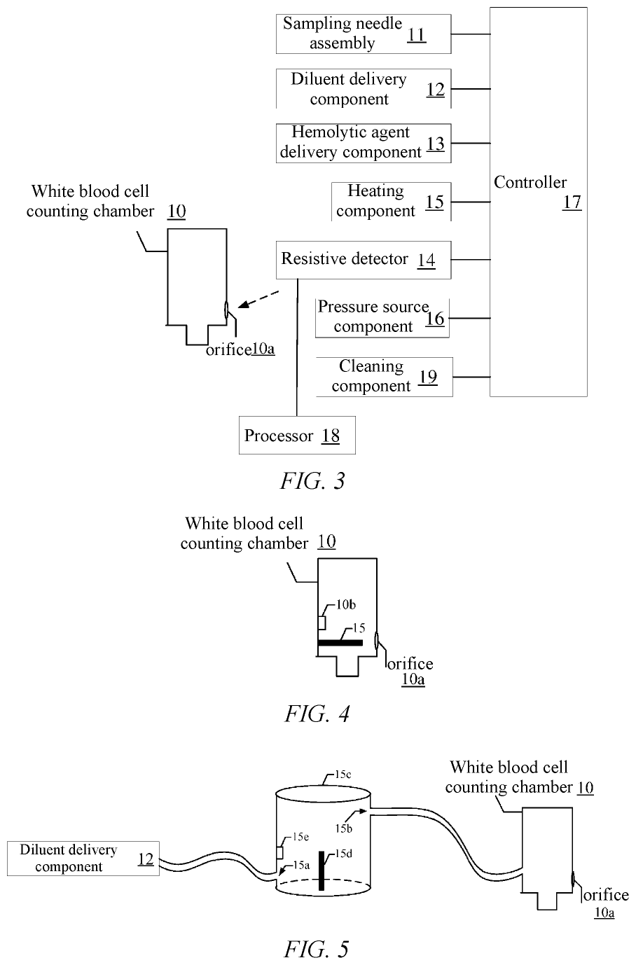

Referring to FIG. 2, an embodiment discloses a cell analyzer, comprising a white blood cell counting chamber 10 having an orifice 10a, a sampling needle assembly 11, a diluent delivery component 12, a hemolytic agent delivery component 13, a resistive detector 14, a heating component 15, a pressure source component 16, a controller 17 and a processor 18. It can be understood that, the controller 17 and the processor 18 may be integrated in a component having processing function and control function in some examples, and may also be two separate components in some examples. Referring to FIG. 3, the cell analyzer of an embodiment may also comprise a cleaning component 19 for cleaning the white blood cell counting chamber 10.

The sampling needle assembly 11 is configured for discharging a sample to be analyzed into the white blood cell counting chamber 10. The diluent delivery component 12 is configured for delivering a diluent to the white blood cell counting chamber 10. The hemolytic agent delivery component 13 is configured for delivering a hemolytic agent to the white blood cell counting chamber 10. The heating component 15 is configured for controlling a temperature of the liquid in the white blood cell counting chamber 10. The pressure source component 16 is configured for providing pressure to enable the liquid in the white blood cell counting chamber 10 to pass through the orifice 10a. The testing principle of the resistive detector 14 is based on the principle of said impedance method, i.e., testing cells passing through the orifice 10a of the white blood cell counting chamber 10, generating corresponding pulses, and outputting these data to the processor 18.

In an embodiment, the controller 18 is configured to control the diluent delivery component 12 to deliver the diluent to the white blood cell counting chamber 10, control the sampling needle assembly 11 to add the sample to be analyzed to the white blood cell counting chamber 10, control the hemolytic agent delivery component 13 to add the hemolytic agent to the white blood cell counting chamber 10 at least once, and control the heating component 15 to control the temperature of the liquid in the white blood cell counting chamber 10 to be within a preset temperature range; and the controller 18 is further configured to control the pressure source component 16 to provide pressure to enable the liquid in the white blood cell counting chamber 10 to pass through the orifice 10a, and control the resistive detector 14 to test the liquid passing through the orifice 10a. The processor 18 is configured to perform at least four-classification and counting of white blood cells according to data output by the resistive detector 14.

It can be seen that the sample, the diluent and the hemolytic agent are added to the white blood cell counting chamber through the sampling needle assembly 11, the diluent delivery component 12 and the hemolytic agent delivery component 13, thereby preparing a sample liquid treated with the hemolytic agent for testing; and then the pressure source component 16 provides pressure such that the liquid in the white blood cell counting chamber 10 passes through the orifice 10a, and the resistive detector 14 tests the liquid passing through the orifice 10a. During this process, the heating component 15 controls the temperature of the liquid in the white blood cell counting chamber 10 to be within the preset temperature range. For example, during the preparation of the sample liquid for testing and/or during the testing, the temperature of the liquid in the white blood cell counting chamber 10 is controlled to be within the preset temperature range, in order to make various types of white blood cells are more obvious in volume difference and more differentiable under the action of the hemolytic agent, so as to finally achieve relatively accurate four-classification or even five-classification of white blood cells. The heating component 15 controls the temperature of the liquid in the white blood cell counting chamber 10 within the preset temperature range. There are many implementation solutions, some of which will be listed below.

Referring to FIG. 4, in an embodiment, the white blood cell counting chamber 10 is provided with a temperature sensor 10b, and the temperature sensor 10b is configured for measuring the temperature of the liquid in the white blood cell counting chamber 10. The heating component 15 is arranged in the white blood cell counting chamber 10, for example, the heating component 15 is a heating rod that generates heat after being energized, and is configured for heating the liquid in the white blood cell counting 10. Specifically, the controller 17 controls the heating component 15 for heating when it is determined that the temperature of the liquid in the white blood cell counting chamber 10 is lower than the preset temperature range according to the data of the temperature sensor 10b, for example, when the preset temperature range is from T1 to T2 and it is determined that the temperature of the liquid in the white blood cell counting chamber 10 is lower than T1. Accordingly, the controller 17 controls the heating component 15 to stop heating when it is determined that the temperature of the liquid in the white blood cell counting chamber 10 is higher than the preset temperature range, for example, when it is determined that the temperature of the liquid in the white blood cell counting chamber 10 is higher than T2.

Referring to FIG. 5, in an embodiment, the heating component 15 comprises a container 15c having a liquid inlet 15a and a liquid outlet 15b; and a heating member 15d for heating the liquid in the container and a temperature sensor 15e for measuring the temperature of the liquid in the container 15c, which are arranged in the container 15c. The liquid inlet 15a is in communication with the diluent delivery component 12 through a pipeline, and the liquid outlet 15b is connected to the white blood cell counting chamber 10 through a pipeline. In this way, the diluent delivered by the diluent delivery component 12 enters the container 15c via the liquid inlet 15a, and flows out of the container 15c via the liquid outlet 15b and then enters the white blood cell counting chamber 10. The controller 17 controls the heating member 15d for heating when it is determined that the temperature of the diluent in the container 15c is lower than a first temperature according to the data of the temperature sensor 15e, and controls the heating member 15d to stop heating when it is determined that the temperature of the diluent in the container 15c is higher than a second temperature. For example, the heating component 15 controls the temperature of the liquid in the white blood cell counting chamber 10 to be within the preset temperature range. If the preset temperature range is from T1 to T2, the first temperature may be T1 or T1−1 degrees Celsius, and the second temperature may be T2 or T2+1 degrees Celsius.

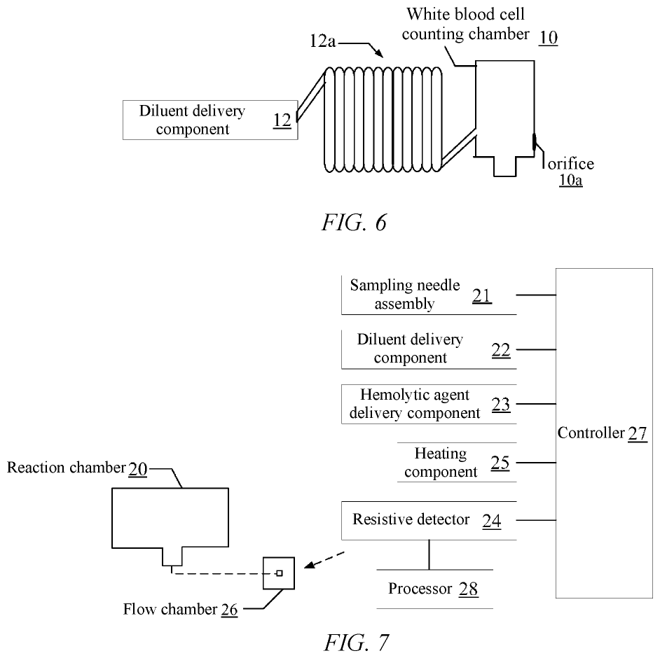

Referring to FIG. 6, in an embodiment, the diluent delivery component 12 is provided with a helical pipeline 12a in communication with the white blood cell counting chamber 10, i.e., the helical pipeline 12a is a pipeline through which the diluent delivery component 12 delivers the diluent to the white blood cell counting chamber 10. The helical pipeline 12a is provided with the heating component 15 (not shown in the figures), i.e., the heating component 15 is arranged at the helical pipeline 12a. For example, the heating component 15 may be an electrothermal film and arranged at an outer wall or an inner wall of the helical pipeline 12a, etc. In this way, the heating component 15 can heat the diluent flowing through the helical pipeline 12a. For example, under the control of the controller 17, the heating component 15 heats the diluent in the helical pipeline 12a that flows to the white blood cell counting chamber 10, such that the temperature of the liquid in the white blood cell counting chamber 10 is controlled to be within the preset temperature range. Specifically, a temperature sensor 10b may also be provided in the white blood cell counting chamber 10. The controller 17 controls the heating component 15 for heating when it is determined that the temperature of the liquid in the white blood cell counting chamber 10 is lower than the preset temperature range according to the data of the temperature sensor 10b, for example, when the preset temperature range is from T1 to T2 and it is determined that the temperature of the liquid in the white blood cell counting chamber 10 is lower than T1. Accordingly, the controller 17 controls the heating component 15 to stop heating when it is determined that the temperature of the liquid in the white blood cell counting chamber 10 is higher than the preset temperature range, for example, when it is determined that the temperature of the liquid in the white blood cell counting chamber 10 is higher than T2.

Several implementation solutions of the heating component 15 are exemplified above, and the following will describe how to treat and test a sample.

In an embodiment, the controller 17 controls the hemolytic agent delivery component 13 to add the hemolytic agent to the white blood cell counting chamber 10 only once, such that an amount of red blood cell fragments in the sample is less than a preset threshold, and thus the counting of white blood cells will not be influenced when the sample is tested. In addition, in the preset temperature range, various types of white blood cells will shrink under the action of the hemolytic agent, such that the difference in size among the various types of white blood cells will be further magnified, and the various types of white blood cells become more obviously and easily distinguished. The controller 17 controls the resistive detector 14 to test the liquid in the white blood cell counting chamber once, and the processor 18 obtains a white blood cell histogram according to the data output by the resistive detector 14 and performs the at least four-classification and counting of white blood cells according to the white blood cell histogram. The vertical axis of the white blood cell histogram represents the cell number, and the horizontal axis represents the cell volume, and then several discrimination lines are provided on the horizontal axis so as to perform classification and counting of white blood cells. In this embodiment, in the preset temperature range, the sample is treated with the hemolytic agent once, and then the at least four-classification and counting of white blood cells can be relatively accurately performed.

In an embodiment, the controller 17 controls the hemolytic agent delivery component 13 to add the hemolytic agent to the white blood cell counting chamber 10 only once. In an example, with this addition of hemolytic agent, the counting of white blood cells will be still influenced by the red blood cell fragments in the sample during a first testing. The controller 17 controls the resistive detector 14 to test the liquid in the white blood cell counting chamber 10 once, and the processor 18 obtains a first white blood cell histogram according to the data output by the resistive detector 14 in this testing, i.e., in the first testing. After waiting for a preset period of time, during which the previously added hemolytic agent continues to act on the sample for further lysing red blood cells such that an amount of red blood cell fragments in the sample is less than a preset threshold, and thus the red blood cell fragments will not influence the counting of white blood cells, and various types of cells in white blood cells continue to shrink in different rates, the controller 17 controls the resistive detector 14 to test the liquid in the white blood cell counting chamber 10 once, and the processor 18 obtains a second white blood cell histogram according to the data output by the resistive detector 14 in this testing, i.e., in a second testing, wherein an amount of red blood cell fragments in the second white blood cell histogram being less than a preset threshold. The processor 18 performs the at least four-classification and counting of white blood cells according to the first white blood cell histogram and the second white blood cell histogram. In this embodiment, in the preset temperature range, the sample is subjected to a first treatment with the hemolytic agent and is then subjected to a first testing, and then after waiting for a preset period of time such that the hemolytic agent continues to act on the red blood cells and the white blood cells in the sample to complete a second treatment on the sample, the sample is subjected to a second testing, thereby relatively accurately realizing the at least four-classification and counting of white blood cells.

In an embodiment, the controller 17 controls the hemolytic agent delivery component 13 to add a first hemolytic agent to the white cell counting chamber; the controller 17 controls the resistive detector 14 to test the liquid in the white blood cell counting chamber 10 once, and the processor 18 obtains a first white blood cell histogram according to the data output by the resistive detector 14 in this testing, i.e., in a first testing; the controller 17 further controls the hemolytic agent delivery component 13 to add a second hemolytic agent to the white cell counting chamber 10; and the controller 17 controls the resistive detector 14 to test the liquid in the white blood cell counting chamber 10 once, the processor 18 obtains a second white blood cell histogram according to the data output by the resistive detector 14 in this testing, i.e., in a second testing, wherein an amount of red blood cell fragments in the second white blood cell histogram is less than a preset threshold, such that the red blood cell fragments will not influence the counting of white blood cells. The processor 18 performs the at least four-classification and counting of white blood cells according to the first white blood cell histogram and the second white blood cell histogram. In this embodiment, in the preset temperature range, the sample is subjected to a first treatment with the first hemolytic agent, is then subjected to a first testing, is then subjected to a second treatment with the second hemolytic agent, and is then subjected to a second testing, thereby relatively accurately realizing the at least four-classification and counting of white blood cells. In an embodiment, the first hemolytic agent and the second hemolytic agent are the same hemolytic agent.

The following will describe how the processor 18 performs the at least four-classification classification and counting of white blood cells according to the first white blood cell histogram and the second white blood cell histogram.

In an embodiment, the processor 18 obtains a percentage of lymphocytes, a percentage of monocytes and a percentage of granulocytes according to the first white blood cell histogram. In some examples, the processor 18 performs data processing on the first white blood cell histogram to remove influence of red blood cell fragments, and obtains the percentage of lymphocytes, the percentage of monocytes and the percentage of granulocytes according to the first white blood cell histogram after removing influence of red blood cell fragments. For example, the processor 60 may obtain a count of white blood cells from the first white blood cell histogram, and may also obtain a count of white blood cells from the second white blood cell histogram, and then calculate a ratio value of the count of white blood cells in the first white blood cell histogram to the count of white blood cells in the second white blood cell histogram. When the ratio value is less than a preset value, the percentage of lymphocytes, the percentage of monocytes and the percentage of granulocytes are directly obtained from the first white blood cell histogram, and when the ratio value is greater than or equal to the preset value, a first discriminator position between the red blood cell fragments and the white blood cells is determined in the first white blood cell histogram according to the ratio value so as to obtain the first white blood cell histogram after removing influence of the red blood cell fragments, and the percentage of lymphocytes, the percentage of monocytes and the percentage of granulocytes are obtained according to the first white blood cell histogram after removing influence of red blood cell fragments. In an embodiment, the first discriminator position between the red blood cell fragments and the white blood cells satisfies the following relationship: an area ratio of a total area of the first white blood cell histogram to an area of the histogram to the right of the first discriminator is equal to the ratio value. In an embodiment, the preset value is approximately 1.02.

In an embodiment, the processor 18 obtains a count of white blood cells, a percentage of eosinophils and a count of eosinophils according to the second white blood cell histogram. It should be noted that, the actually obtained eosinophils include basophils, but since the number of the basophils is very small compared with that of the eosinophils, it can be considered that the cells obtained at this time are eosinophils. In this way, the processor 18 subtracts the percentage of eosinophils from the percentage of granulocytes to obtain a percentage of neutrophils; and the processor 18 may calculate a count of lymphocytes, a count of monocytes and a count of neutrophils according to the count of white blood cells, the percentage of lymphocytes, the percentage of monocytes and the percentage of neutrophils. For example, the count of white blood cells obtained from the second white blood cell histogram is taken as the count of white blood cells in four-classification parameters; the percentage of lymphocytes obtained from the first white blood cell histogram is taken as the percentage of lymphocytes in the four-classification parameters, and the percentage of lymphocytes obtained from the first white blood cell histogram is multiplied by the count of white blood cells obtained from the second white blood cell histogram to obtain the count of lymphocytes, which is taken as the count of lymphocytes in the four-classification parameters; the percentage of monocytes obtained from the first white blood cell histogram is taken as the percentage of monocytes in the four-classification parameters, and the percentage of monocytes obtained from the first white blood cell histogram is multiplied by the count of white blood cells obtained from the second white blood cell histogram to obtain the count of monocytes, which is taken as the count of monocytes in the four-classification parameters; the percentage of eosinophils obtained from the second white blood cell histogram is subtracted from the percentage of granulocytes obtained from the first white blood cell histogram to obtain the percentage of neutrophils, which is taken as the percentage of neutrophils in the four-classification parameters, and the percentage of neutrophils is multiplied by the count of white blood cells obtained from the second white blood cell histogram to obtain the count of neutrophils, which is taken as the count of neutrophils in the four-classification parameters; and the percentage of eosinophils and the count of eosinophils obtained from the second white blood cell histogram are taken as the percentage of eosinophils and the count of eosinophils in the four-classification parameters. In this way, the four-classification and counting of white blood cells are completed.

In an embodiment, the processor 18 obtains a count of white blood cells, a percentage of basophils and a count of basophils according to the second white blood cell histogram. In this way, the processor 18 subtracts the percentage of basophils from the percentage of granulocytes to obtain a percentage of a total of neutrophils and eosinophils; and the processor 18 can calculate a count of lymphocytes, a count of monocytes, and a total count of neutrophils and eosinophils, according to the count of white blood cells, the percentage of lymphocytes, the percentage of monocytes, and the percentage of the total of neutrophils and eosinophils. For example, the count of white blood cells obtained from the second white blood cell histogram is taken as the count of white blood cells in four-classification parameters; the percentage of lymphocytes obtained from the first white blood cell histogram is taken as the percentage of lymphocytes in the four-classification parameters, and the percentage of lymphocytes obtained from the first white blood cell histogram is multiplied by the count of white blood cells obtained from the second white blood cell histogram to obtain the count of lymphocytes, which is taken as the count of lymphocytes in the four-classification parameters; the percentage of monocytes obtained from the first white blood cell histogram is taken as the percentage of monocytes in the four-classification parameters, and the percentage of monocytes obtained from the first white blood cell histogram is multiplied by the count of white blood cells obtained from the second white blood cell histogram to obtain the count of monocytes, which is taken as the count of monocytes in the four-classification parameters; the percentage of basophils obtained from the second white blood cell histogram is subtracted from the percentage of granulocytes obtained from the first white blood cell histogram to obtain the percentage of the total of neutrophils and eosinophils, which is taken as the percentage of the total of neutrophils and eosinophils in the four-classification parameters, and the percentage of the total of neutrophils and eosinophils is multiplied by the count of white blood cells obtained from the second white blood cell histogram to obtain the total count of neutrophils and eosinophils, which is taken as the total count of neutrophils and eosinophils in the four-classification parameters; and the percentage of basophils and the count of basophils obtained from the second white blood cell histogram are taken as the percentage of basophils and the count of basophils in the four-classification parameters. In this way, the four-classification and counting of white blood cells are completed.

In an embodiment, the processor 18 obtains a count of white blood cells, a percentage of basophils, a count of basophils, a percentage of eosinophils and a count of eosinophils, according to the second white blood cell histogram. In this way, the processor 18 subtracts the percentage of basophils and the percentage of eosinophils from the percentage of granulocytes to obtain a percentage of neutrophils; and the processor 18 can calculate a count of lymphocytes, a count of monocytes and a count of neutrophils according to the count of white blood cells, the percentage of lymphocytes, the percentage of monocytes and the percentage of neutrophils. For example, the count of white blood cells obtained from the second white blood cell histogram is taken as the count of white blood cells in five-classification parameters; the percentage of lymphocytes obtained from the first white blood cell histogram is taken as the percentage of lymphocytes in the five-classification parameters, and the percentage of lymphocytes obtained from the first white blood cell histogram is multiplied by the count of white blood cells obtained from the second white blood cell histogram to obtain the count of lymphocytes, which is taken as the count of lymphocytes in the five-classification parameters; the percentage of monocytes obtained from the first white blood cell histogram is taken as the percentage of monocytes in the five-classification parameters, and the percentage of monocytes obtained from the first white blood cell histogram is multiplied by the count of white blood cells obtained from the second white blood cell histogram to obtain the count of monocytes, which is taken as the count of monocytes in the five-classification parameters; the percentage of basophils and the percentage of eosinophils obtained from the second white blood cell histogram is subtracted from the percentage of granulocytes obtained from the first white blood cell histogram to obtain the percentage of neutrophils, which is taken as the percentage of neutrophils in the five-classification parameters, and the percentage of neutrophils is multiplied by the count of white blood cells obtained from the second white blood cell histogram to obtain the count of neutrophils, which is taken as the count of neutrophils in the five-classification parameters; the percentage of basophils and the count of basophils obtained from the second white blood cell histogram are taken as the percentage of basophils and the count of basophils in the five-classification parameters; and the percentage of eosinophils and the count of eosinophils obtained from the second white blood cell histogram are taken as the percentage of eosinophils and the count of eosinophils in the four-classification parameters. In this way, the five-classification and counting of white blood cells are completed.

Example of treating and testing the same portion of a blood sample or a sample are described above, and it is also possible to perform treatment and testing on two separate portions of a sample, i.e., a blood sample, which will be described in detail below.

In an embodiment, the controller 17 controls the diluent delivery component 12 to deliver a diluent to the white blood cell counting chamber 10; the controller 17 controls the sampling needle assembly 11 to aspirate a sample to be analyzed, and discharge a portion of the sample to the white blood cell counting chamber 10; the controller 17 controls the hemolytic agent delivery component 13 to add a first hemolytic agent to the white blood cell counting chamber, i.e., for treating a first blood portion; the controller 17 controls the pressure source component 16 to provide pressure to enable the liquid in the white blood cell counting chamber 10 to pass through the orifice 10a, and controls the resistive detector 14 to test the liquid passing through the orifice, and the processor 18 obtains a first white blood cell histogram according to the data output by the resistive detector in this testing, i.e., in the testing on the first blood portion; the controller 17 controls the white blood cell counting chamber 10 to discharge liquid, and controls the cleaning component 19 to clean the white blood cell counting chamber 10; the controller 17 controls the diluent delivery component 12 to deliver a diluent to the white blood cell counting chamber 10; the controller 17 controls the sampling needle assembly 11 to discharge at least a portion of the remaining sample to the white blood cell counting chamber 10; the controller 17 controls the hemolytic agent delivery component 13 to add a second hemolytic agent to the white blood cell counting chamber 10, i.e., for treating a second blood portion; and the controller 17 controls the pressure source component 16 to provide pressure to enable the liquid in the white blood cell counting chamber 10 to pass through the orifice 10a, and controls the resistive detector 14 to test the liquid passing through the orifice 10a. The processor 18 obtains a second white blood cell histogram according to the data output by the resistive detector 14 in this testing, i.e., in the testing on the second blood portion, wherein an amount of red blood cell fragments in the second white blood cell histogram is less than a preset threshold. The controller 17 controls the heating component 15 to control the temperature of the liquid in the white blood cell counting chamber to be within the preset temperature range before each testing. The processor 18 performs at least four-classification and counting of white blood cells according to the first white blood cell histogram and the second white blood cell histogram. For specific process, reference can be made to the above description of how the processor 18 performs at least four-classification and counting of white blood cells according to the first white blood cell histogram and the second white blood cell histogram in the testing on the same blood sample portion, which will not be repeated here. In an embodiment, a dosage of the first hemolytic agent is less than a dosage of the second hemolytic agent, wherein the dosage of the first hemolytic agent is set such that there are still red blood cell fragments, which affect the counting of white blood cells, in the first blood portion during the testing, and the dosage of the second hemolytic agent is set such that an amount of red blood cell fragments in the second blood portion is less than a preset threshold, i.e., there are no red blood cell fragments that affect the counting of white blood cells. In an embodiment, the first hemolytic agent and the second hemolytic agent are of a same type of hemolytic agent.

The above are some descriptions of the cell analyzer using an electrical impedance method, and the disclosure also discloses a cell analyzer using an impedance method based on sheath flow principle in some examples, which will be described in detail below.

Referring to FIG. 7, in an embodiment, the cell analyzer comprises a reaction chamber 20, a sampling needle assembly 21, a diluent delivery component 22, a hemolytic agent delivery component 23, a resistive detector 24, a heating component 25, a flow chamber 26, a controller 27 and a processor 28. It can be understood that the controller 27 and the processor 28 may be integrated in a component having processing and control functions in some examples, and may also be two separate components in some examples.

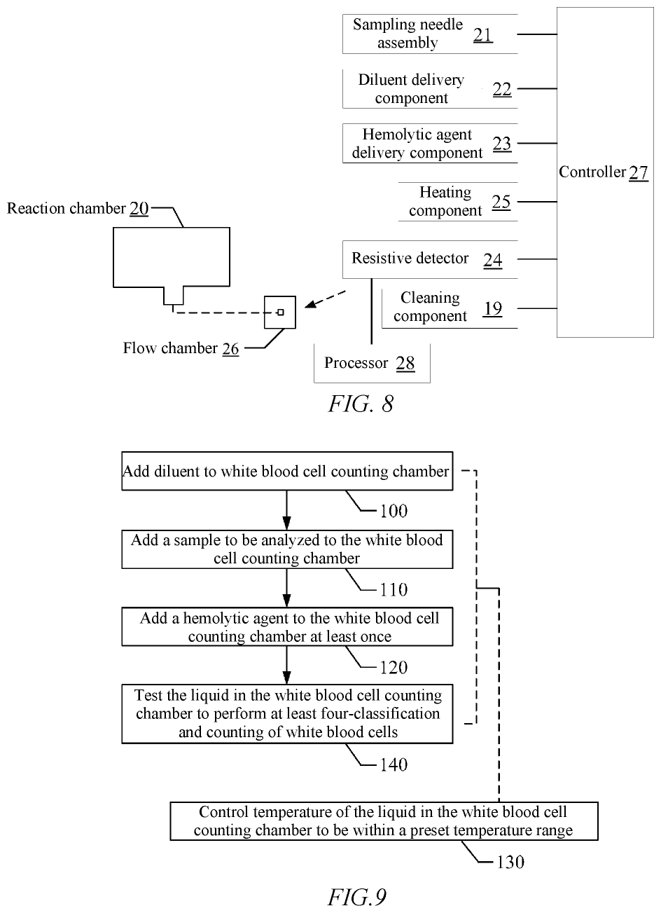

Referring to FIG. 8, the cell analyzer of an embodiment may also comprise a cleaning component 29 for cleaning the reaction chamber 20. The sampling needle assembly 21 is configured for discharging a sample to be analyzed into the reaction chamber 20. The diluent delivery component 12 is configured for delivering a diluent to the reaction chamber 20. The hemolytic agent delivery component 13 is configured for delivering a hemolytic agent to the reaction chamber 20. The heating component 15 is configured for controlling a temperature of the liquid in the reaction chamber 20. The flow chamber 26 is configured for cells in the sample to be analyzed to pass through one by one. The principle of testing by the resistive detector 24 is based on the principle of the impedance method, i.e., testing the cells passing through the flow chamber 26, generating corresponding pulses, and outputting the data to the processor 18. The structure and working process of the heating component 25 can be referred to the heating component 15, which will not be repeated here.

The following describes an example of treating and testing a same portion of a blood sample or a sample by means of the cell analyzer using the impedance method based on the sheath flow principle. In an embodiment, the controller 27 controls the diluent delivery component 22 to deliver a diluent to the reaction chamber 20; the controller 27 controls the sampling needle assembly 21 to add a sample to be analyzed to the reaction chamber 10; the controller 27 controls the hemolytic agent delivery component 23 to add a hemolytic agent to the reaction chamber 10 at least once; the controller 27 controls the heating component 25 to control a temperature of the liquid in the reaction chamber 10 within a preset temperature range; the controller 27 controls cells in the liquid in the reaction chamber 20 to pass through the flow chamber 26 one by one, and controls the resistive detector 24 to test the cells passing through the flow chamber 26; and the processor 28 performs at least four-classification and counting of white blood cells according to the data output by the resistive detector 24. In some examples, the cell analyzer may treat the sample with the hemolytic agent in the preset temperature range once, so that the at least four-classification and counting of white blood cells can thus be performed relatively accurately. In some examples, the cell analyzer may perform a first treatment on the sample with the hemolytic agent in the preset temperature range and then perform a first testing, and then wait for a preset period of time such that the hemolytic agent continues to act on red blood cells and white blood cells in the sample to complete a second treatment on the sample, and then perform a second testing, thereby relatively accurately realizing the at least four-classification and counting of white blood cells. In some examples, the cell analyzer may perform a first treatment on the sample with a first hemolytic agent in the preset temperature range and then perform a first testing, then perform a second treatment on the sample with a second hemolytic agent and then perform a second testing, thereby relatively accurately realizing the at least four-classification and counting of white blood cells. The specific process can be referred to the description of the cell analyzer in FIG. 2, which will not be repeated here.

The following describes an example of treating and testing two blood portions of a sample, i.e., a blood sample by means of the cell analyzer using the impedance method based on the sheath flow principle. In an embodiment, the controller 27 controls the diluent delivery component 22 to deliver a diluent to the reaction chamber 20; the controller 27 controls the sampling needle assembly 21 to aspirate a sample from a sample to be analyzed, and discharge a portion of the sample to the reaction chamber 20; the controller 27 controls the hemolytic agent delivery component 23 to add a first hemolytic agent to the reaction chamber 20, i.e., for treating a first blood portion; the controller 27 controls the liquid in the reaction chamber 20 to pass through the flow chamber 26, and controls the resistive detector 24 to test the cells passing through the flow chamber 26, i.e., tests the first blood portion, and the processor 28 obtains a first white blood cell histogram according to the data output by the resistive detector 24 in this testing, i.e., in the testing on the first blood portion; the controller 27 controls the reaction chamber 20 to discharge liquid, and controls the cleaning component 29 to clean the reaction chamber 20; the controller 27 controls the diluent delivery component 22 to deliver a diluent to the reaction chamber 20; the controller 27 controls the sampling needle assembly 21 to discharge at least a portion of the remaining sample to the reaction chamber 20; the controller 27 controls the hemolytic agent delivery component 23 to add a second hemolytic agent to the reaction chamber 20, i.e., for treating a second blood portion; and the controller 27 controls the liquid in the reaction chamber 20 to pass through the flow chamber 26, and controls the resistive detector 24 to test the cells passing through the flow chamber 26, i.e., tests the second blood portion, and the processor 28 obtains a second white blood cell histogram according to the data output by the resistive detector 24 in this testing, i.e., in the testing on the second blood portion, wherein an amount of red blood cell fragments in the second white blood cell histogram is less than a preset threshold. The controller 27 controls the heating component to control the temperature of the liquid in the reaction chamber 20 within the preset temperature range before each testing. The processor 28 performs at least four-classification and counting of white blood cells according to the first white blood cell histogram and the second white blood cell histogram. The specific process can be referred to how the processor 18 performs at least four-classification and counting of white blood cells according to the first white blood cell histogram and the second white blood cell histogram in the testing on a blood sample portion described in the above, which will not be repeated here. In an embodiment, a dosage of the first hemolytic agent is less than a dosage of the second hemolytic agent, the dosage of the first hemolytic agent is set such that there are still red blood cell fragments, which affect the counting of white blood cells, in the first blood portion during the testing, and the dosage of the second hemolytic agent is set such that an amount of red blood cell fragments in the second blood portion is less than a preset threshold, i.e., there are no red blood cell fragments that s affect the counting of white blood cells. In an embodiment, the first hemolytic agent and the second hemolytic agent are a same hemolytic agent.

In the cell analyzer of the above embodiments, the processed sample or blood sample may be an animal's blood sample, multiple animal modes may be preset in the cell analyzer, and the cell analyzer (for example, its controller) selects, in response to a user's instruction of selecting a mode, a corresponding animal mode from the multiple animal modes, in which each animal mode has a corresponding hemolytic agent and dosage and a preset temperature range; and then tests the animal's blood sample according to the selected animal mode. In an embodiment, the animal modes include one or both of a feline mode and a canine mode, in which the preset temperature range corresponding to the feline mode is 31 degrees Celsius to 40 degrees Celsius, and the preset temperature range corresponding to the canine mode is 28 degrees Celsius to 38 degrees Celsius, preferably, the preset temperature range corresponding to the feline mode and the canine mode is about 35 degrees Celsius.

In the disclosure, any suitable method may be used to perform three-classification based on the first white blood cell histogram. For example, as shown in FIG. 12A, firstly, a first boundary 1 between a first type of white blood cells and a second type of white blood cells is determined according to a trough point C between two peak points A and B in the first white blood cell histogram, wherein the first type of white blood cells is smaller in volume than the second type of white blood cells, and the area in first white blood cell histogram, in which the volume is smaller than the volume represented by the first boundary 1, represents the first type of white blood cells (for example, lymphocytes (LYM) as shown in FIG. 12A).

The two peak points A and B in the first white blood cell histogram can be firstly determined, and the trough point C can be determined by any suitable method, for example, the trough point C is the minimum point corresponding to the minimum ordinate value between the peak point A and the peak point B.

Next, as shown in FIG. 12A, a second boundary 2 between the second type of white blood cells (such as monocytes (MON)) and a third type of white blood cells (such as granulocytes (NEU)) is determined according to the first boundary 1, wherein the second boundary 2 and the first boundary 1 are separated by a first predetermined volume, and the volume corresponding to the second boundary is greater than the volume corresponding to the first boundary.

The first predetermined volume may be reasonably set according to prior experience. For example, under specific reaction conditions, reaction temperatures, and dosage of reagents (including the hemolytic agent and the diluent), the volume between the trough point and the actual second boundary 2 may be obtained through multiple tests under these specific conditions, in particular under the specific usage of hemolytic agent, thereby determining the first predetermined volume, wherein under different reaction conditions, reaction temperatures, and dosage of reagents (including the hemolytic agent and the diluent), the position of the trough point and the first predetermined volume value may also be different, which may be reasonably adjusted according to actual situations.

Any suitable method may be used for classification from the second white blood cell histogram. For example, as shown in FIG. 12B, starting from the maximum volume Vmax (for example, Vmax=250 fL) of the second white blood cell histogram, a second critical point D, where the slope of the curve in the second white blood cell histogram is greater than a second threshold slope K for the first time, is sought in a volume decreasing direction.

The maximum volume Vmax may refer to the end position of the white blood cells in the white blood cell histogram. In the curve in the second white blood cell histogram, the slopes of the points on a predetermined segment of the curve starting from the maximum volume Vmax in the volume decreasing direction are less than or equal to 0, so the value of the second threshold slope K is set to be less than zero. Specifically, the value of the second threshold slope K may be set according to actual conditions.

Next, a third boundary 3 between the third type of white blood cells and a fourth type of white blood cells is determined based on the second critical point D, and the third boundary 3 is a straight line that passes through the second critical point D and is perpendicular to the horizontal axis of the second white blood cell histogram, thereby achieving a four-classification of the white blood cells. The area between the third boundary 3 and Vmax represents the fourth type of white blood cells (such as eosinophils (EOS), although in fact the area between the third boundary 3 and Vmax represents eosinophils (EOS) and basophils (BASO), the number of the basophils (BASO) is relatively small with respect to that of the eosinophils (EOS), so the cells in this area can all be considered as eosinophils (EOS), and then the eosinophils (EOS) obtained from the second histogram is subtracted from the granulocytes (NEU) obtained from the first histogram to obtain neutrophils (NEU)).

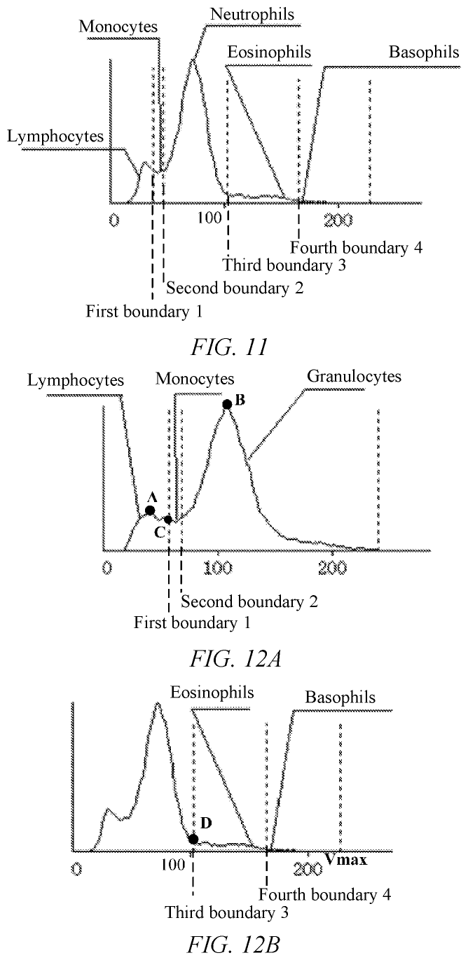

Any suitable method may be used for classification from the second white blood cell histogram. For example, a fourth boundary 4 between the fourth type of white blood cells and a fifth type of white blood cells is determined according to the third boundary 3, wherein the fourth boundary 4 and the third boundary 3 are separated by a second predetermined volume S_{baso}, and the volume corresponding to the fourth boundary 4 is greater than the volume corresponding to the third boundary 3; and the fourth type of white blood cells are in the area of the second white blood cell histogram that is between the third boundary and the fourth boundary, and the fifth type of white blood cells (such as basophils (BASO)) are in the area of the second white blood cell histogram whose volume is greater than the volume corresponding to the fourth boundary 4, i.e., the area between the fourth boundary 4 and the maximum volume Vmax. It can be understood that, as shown in FIG. 11, the white blood cell histogram may also use the above method to perform a four-classification or even a five-classification of white blood cells. For example, a first boundary 1, a second boundary 2, a third boundary 3 and a fourth boundary 4 are respectively determined in FIG. 11 according to the above method; a first type of white blood cells (lymphocytes (LYM)) are in the area of the white blood cell histogram as shown in FIG. 11 whose volume is smaller than the volume represented by the first boundary 1; a second type of white blood cells (such as monocytes (MON)) are in the area between the second boundary 2 and the first boundary 1; a third type of white blood cells (such as neutrophils (NEU)) are in the area between the third boundary 3 and the second boundary 2; a fourth type of white blood cells (such as eosinophils (EOS)) are in the area between the fourth boundary 4 and the third boundary 3; and a fifth type of white blood cells (such as basophils (BASO)) are in the area between the maximum volume Vmax and the fourth boundary 4.

Referring to FIG. 9, an embodiment of the disclosure also discloses a method for classifying white blood cells based on an impedance method, which may include steps 100 to 140, and will be described in detail below.

In step 100, a diluent is added to a white blood cell counting chamber.

In step 110, a sample to be analyzed is added to the white blood cell counting chamber.

In step 120: a hemolytic agent is added to the white blood cell counting chamber at least once.

It can be seen that the operations of adding the diluent, the sample and the hemolytic agent to the white blood cell counting chamber are completed in steps 100 to 120, with the purpose of treating the sample with the hemolytic agent. It can be understood that these steps are only for a clear description of an embodiment and are not meant to be a necessary order, and they can be exchanged or adjusted in a manner that is understood by those skilled in the art.

In step 130, a temperature of the liquid in the white blood cell counting chamber is controlled to be within a preset temperature range. In step 130, there are many implementation solutions to control the temperature of the liquid in the white blood cell counting chamber to be within the preset temperature range. For example, in an embodiment, step 130 may comprise: heating the diluent to a certain temperature, and then adding the diluent to the white blood cell counting chamber so as to control the temperature of the liquid in the white blood cell counting chamber to be within the preset temperature range. For example, in an embodiment, step 130 may also comprise: heating the liquid in the white blood cell counting chamber so as to control the temperature of the liquid in the white blood cell counting chamber to be within the preset temperature range.

In step 140, the liquid in the white blood cell counting chamber is tested to perform at least four-classification and counting of white blood cells.

The following describes how to treat the sample in step 120 and how to test the sample in step 140.

In an embodiment, the sample may be treated with the hemolytic agent in the preset temperature range once, and then the at least four-classification classification and counting of white blood cells can be relatively accurately performed. For example, in an embodiment, in step 120, the hemolytic agent is added to the white blood cell counting chamber only once, such that an amount of red blood cell fragments in the sample is less than a preset threshold, and thus the counting of white blood cells will not be influenced when the sample is tested. In addition, in the preset temperature range, various types of cells in the white blood cells will shrink under the action of the hemolytic agent, such that such that the difference in volume size among the various types of cells will be further magnified, and the various types of cells become more obviously and easily distinguished. In step 140, the liquid in the white blood cell counting chamber is tested once to obtain a white blood cell histogram; and the at least four-classification classification and counting of white blood cells is performed according to the white blood cell histogram obtained from this testing.

In an embodiment, in the preset temperature range, the sample may be subjected to a first treatment with a first hemolytic agent, then subjected to a first testing, then subjected to a second treatment with a second hemolytic agent, and then subjected to a second testing, thereby relatively accurately realizing the at least four-classification and counting of white blood cells. For example, in an embodiment, in step 120, the first hemolytic agent is added to the cell counting chamber, and then in step 140, the liquid in the white blood cell counting chamber is tested once to obtain a first white blood cell histogram; in step 120, the second hemolytic agent is added to the cell counting chamber such that an amount of red blood cell fragments in the sample is less than a preset threshold such that the red blood cell fragments will not influence the counting of white blood cells; in step 140, the liquid in the white blood cell counting chamber is tested once to obtain a second white blood cell histogram; and the at least four-classification and counting of white blood cells are performed according to the first white blood cell histogram and the second white blood cell histogram. In an embodiment, the first hemolytic agent and the second hemolytic agent are a same hemolytic agent.

In an embodiment, in the preset temperature range, the sample may be subjected to a first treatment with the hemolytic agent and then subjected to a first testing, and then after waiting for a preset period of time such that the hemolytic agent continues to act on the red blood cells and the white blood cells in the sample to complete a second treatment on the sample, the sample may be subjected to a second testing, thereby relatively accurately realizing the at least four-classification and counting of white blood cells. For example, in an embodiment, in step 120, the hemolytic agent is added to the white blood cell counting chamber only once, and in step 140, the liquid in the white blood cell counting chamber is tested once to obtain a first white blood cell histogram; in step 120, after waiting for a preset period of time such that the hemolytic agent continues to act on the sample, the amount of red blood cell fragments in the sample is less than the preset threshold, and in step 140, the liquid in the white blood cell counting chamber is tested once to obtain a second white blood cell histogram; and the at least four-classification and counting of white blood cells are performed according to the first white blood cell histogram and the second white blood cell histogram.

The following describes how to perform the at least four-classification and counting of white blood cells according to the first white blood cell histogram and the second white blood cell histogram in step 140.

In an embodiment, in step 140, data processing is performed on the first white blood cell histogram to remove influence of red blood cell fragments, and a percentage of lymphocytes, a percentage of monocytes and a percentage of granulocytes are obtained according to the first white blood cell histogram after removing influence of the red blood cell fragments. For example, in step 140, a count of white blood cells may be obtained from the first white blood cell histogram, a count of white blood cells may also be obtained from the second white blood cell histogram, and then a ratio value of the count of white blood cells based on the first white blood cell histogram to the count of white blood cells based on the second white blood cell histogram is calculated. When the ratio value is less than a preset value, the percentage of lymphocytes, the percentage of monocytes and the percentage of granulocytes are directly obtained from the first white blood cell histogram, and when the ratio value is greater than or equal to the preset value, a first discriminator position between the red blood cell fragments and the white blood cells is determined in the first white blood cell histogram according to the ratio value so as to obtain the first white blood cell histogram after removing influence of the red blood cell fragments, and the percentage of lymphocytes, the percentage of monocytes and the percentage of granulocytes are obtained according to the first white blood cell histogram after the removal of the influence of the red blood cell fragments. In an embodiment, the first discriminator position between the red blood cell fragments and the white blood cells satisfies the following relationship: an area ratio of a total area of the first white blood cell histogram to an area of the histogram to the right of the first discriminator is equal to the ratio value. In an embodiment, the preset value is approximately 1.02.

In an embodiment, in step 140, a count of white blood cells, a percentage of eosinophils and a count of eosinophils are obtained according to the second white blood cell histogram. It should be noted that, the actually obtained eosinophils include basophils, but since the number of basophils is very small compared with that of the eosinophils, it can be considered that the cells obtained at this time are eosinophils. In this way, in step 140, the percentage of eosinophils is subtracted from the percentage of granulocytes to obtain a percentage of neutrophils; and in step 140, a count of lymphocytes, a count of monocytes and a count of neutrophils may be calculated according to the count of white blood cells, the percentage of lymphocytes, the percentage of monocytes and the percentage of neutrophils. For example, the count of white blood cells obtained from the second white blood cell histogram is taken as the count of white blood cells in four-classification parameters; the percentage of lymphocytes obtained from the first white blood cell histogram is taken as the percentage of lymphocytes in the four-classification parameters, and the percentage of lymphocytes obtained from the first white blood cell histogram is multiplied by the count of white blood cells obtained from the second white blood cell histogram to obtain the count of lymphocytes, which is taken as the count of lymphocytes in the four-classification parameters; the percentage of monocytes obtained from the first white blood cell histogram is taken as the percentage of monocytes in the four-classification parameters, and the percentage of monocytes obtained from the first white blood cell histogram is multiplied by the count of white blood cells obtained from the second white blood cell histogram to obtain the count of monocytes, which is taken as the count of monocytes in the four-classification parameters; the percentage of eosinophils obtained from the second white blood cell histogram is subtracted from the percentage of granulocytes obtained from the first white blood cell histogram to obtain the percentage of neutrophils, which is taken as the percentage of neutrophils in the four-classification parameters, and the percentage of neutrophils is multiplied by the count of white blood cells obtained from the second white blood cell histogram to obtain the count of neutrophils, which is taken as the count of neutrophils in the four-classification parameters; and the percentage of eosinophils and the count of eosinophils obtained from the second white blood cell histogram are taken as the percentage of eosinophils and the count of eosinophils in the four-classification parameters. In this way, the four-classification and counting of white blood cells are completed.

In an embodiment, in step 140, a count of white blood cells, a percentage of basophils and a count of basophils are obtained according to the second white blood cell histogram. In this way, in step 140, the percentage of basophils is subtracted from the percentage of granulocytes to obtain a percentage of a total of neutrophils and eosinophils; and in step 140, a count of lymphocytes, a count of monocytes, and a count of the total of neutrophils and eosinophils may be calculated according to the count of white blood cells, the percentage of lymphocytes, the percentage of monocytes, and the percentage of the total of neutrophils and eosinophils. For example, the count of white blood cells obtained from the second white blood cell histogram is taken as the count of white blood cells in four-classification parameters; the percentage of lymphocytes obtained from the first white blood cell histogram is taken as the percentage of lymphocytes in the four-classification parameters, and the percentage of lymphocytes obtained from the first white blood cell histogram is multiplied by the count of white blood cells obtained from the second white blood cell histogram to obtain the count of lymphocytes, which is taken as the count of lymphocytes in the four-classification parameters; the percentage of monocytes obtained from the first white blood cell histogram is taken as the percentage of monocytes in the four-classification parameters, and the percentage of monocytes obtained from the first white blood cell histogram is multiplied by the count of white blood cells obtained from the second white blood cell histogram to obtain the count of monocytes, which is taken as the count of monocytes in the four-classification parameters; the percentage of basophils obtained from the second white blood cell histogram is subtracted from the percentage of granulocytes obtained from the first white blood cell histogram to obtain the percentage of the total of neutrophils and eosinophils, which is taken as the percentage of the total of neutrophils and eosinophils in the four-classification parameters, and the percentage of the total of neutrophils and eosinophils is multiplied by the count of white blood cells obtained from the second white blood cell histogram to obtain the count of the total of neutrophils and eosinophils, which is taken as the count of the total of neutrophils and eosinophils in the four-classification parameters; and the percentage of basophils and the count of basophils obtained from the second white blood cell histogram are taken as the percentage of basophils and the count of basophils in the four-classification parameters. In this way, the four-classification and counting of white blood cells are completed.

In an embodiment, in step 140, a count of white blood cells, a percentage of basophils, a count of basophils, a percentage of eosinophils and a count of eosinophils are obtained according to the second white blood cell histogram. In this way, in step 140, the percentage of basophils and the percentage of eosinophils are subtracted from the percentage of granulocytes to obtain a percentage of neutrophils; and the processor 18 may calculate a count of lymphocytes, a count of monocytes and a count of neutrophils according to the count of white blood cells, the percentage of lymphocytes, the percentage of monocytes and the percentage of neutrophils. For example, the count of white blood cells obtained from the second white blood cell histogram is taken as the count of white blood cells in five-classification parameters; the percentage of lymphocytes obtained from the first white blood cell histogram is taken as the percentage of lymphocytes in the five-classification parameters, and the percentage of lymphocytes obtained from the first white blood cell histogram is multiplied by the count of white blood cells obtained from the second white blood cell histogram to obtain the count of lymphocytes, which is taken as the count of lymphocytes in the five-classification parameters; the percentage of monocytes obtained from the first white blood cell histogram is taken as the percentage of monocytes in the five-classification parameters, and the percentage of monocytes obtained from the first white blood cell histogram is multiplied by the count of white blood cells obtained from the second white blood cell histogram to obtain the count of monocytes, which is taken as the count of monocytes in the five-classification parameters; the percentage of basophils and the percentage of eosinophils obtained from the second white blood cell histogram is subtracted from the percentage of granulocytes obtained from the first white blood cell histogram to obtain the percentage of neutrophils, which is taken as the percentage of neutrophils in the five-classification parameters, and the percentage of neutrophils is multiplied by the count of white blood cells obtained from the second white blood cell histogram to obtain the count of neutrophils, which is taken as the count of neutrophils in the five-classification parameters; the percentage of basophils and the count of basophils obtained from the second white blood cell histogram are taken as the percentage of basophils and the count of basophils in the five-classification parameters; and the percentage of eosinophils and the count of eosinophils obtained from the second white blood cell histogram are taken as the percentage of eosinophils and the count of eosinophils in the four-classification parameters. In this way, the five-classification and counting of white blood cells are completed.

An example of treating and testing a same portion of a blood sample or a sample is described above, and it is also possible to treat and test two separate blood portions of a sample, i.e., a blood sample, which will be described in detail below.

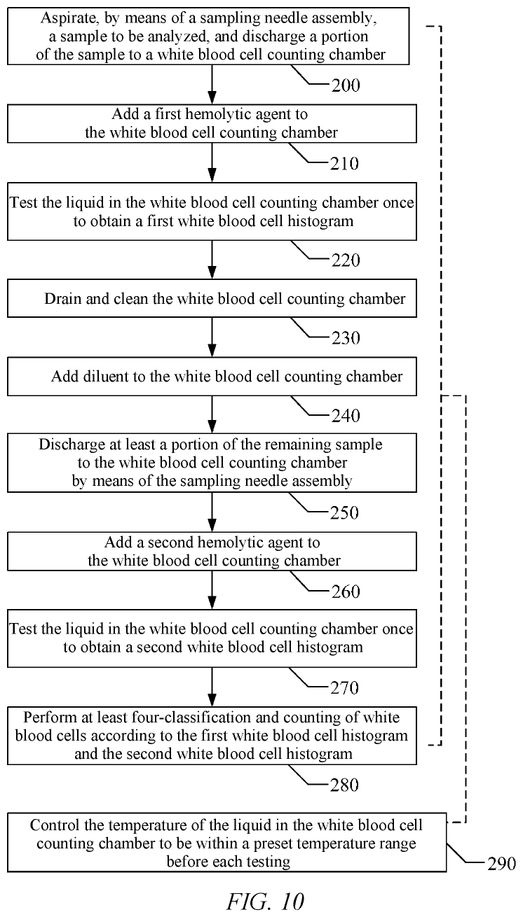

Referring to FIG. 10, an embodiment of the disclosure also discloses a method for classifying white blood cells based on an impedance method, which may include steps 200 to 290, and will be described in detail below.

In step 200, a sampling needle assembly aspirates a sample from a sample to be analyzed, and discharges a portion of the sample to a white blood cell counting chamber.

In step 210, a first hemolytic agent is added to the white blood cell counting chamber, i.e., for treating a first blood portion.

In step 220, the liquid in the white blood cell counting chamber is tested once, i.e., the first blood portion is tested, so as to obtain a first white blood cell histogram.

In step 230, the white blood cell counting chamber is drained and cleaned.

In step 240, diluent is added to a white blood cell counting chamber.

In step 250, the sampling needle assembly discharges at least a portion of the remaining sample to the white blood cell counting chamber.

In step 260, a second hemolytic agent is added to the white blood cell counting chamber, i.e., for treating a second blood portion. In an embodiment, a dosage of the first hemolytic agent is less than a dosage of the second hemolytic agent, the dosage of the first hemolytic agent is set such that there are still red blood cell fragments, which affect the counting of white blood cells, in the first blood portion during the testing, and the dosage of the second hemolytic agent is set such that an amount of red blood cell fragments in the second blood portion is less than a preset threshold, i.e., there are no red blood cell fragments that affect the counting of white blood cells. In an embodiment, the first hemolytic agent and the second hemolytic agent are a same hemolytic agent.

In step 270, the liquid in the white blood cell counting chamber is tested once, i.e., the second blood portion is tested, so as to obtain a second white blood cell histogram.

In step 280, at least four-classification and counting of white blood cells is performed according to the first white blood cell histogram and the second white blood cell histogram, wherein an amount of red blood cell fragments in the second white blood cell histogram is less than a preset threshold. Step 280 can refer to step 140 mentioned above, which will not be repeated here.

In step 290, the temperature of the liquid in the white blood cell counting chamber is controlled to be within a preset temperature range before each testing. For example, step 290 may comprise: heating the diluent to a certain temperature, and then adding the diluent to the white blood cell counting chamber, such that the temperature of the liquid in the white blood cell counting chamber is controlled to be within the preset temperature range; or heating the liquid in the white blood cell counting chamber, such that the temperature of the liquid in the white blood cell counting chamber is controlled to be within the preset temperature range.

In the methods for classifying white blood cells based on an impedance method according to various embodiments of the disclosure, the sample or blood sample to be treated may be an animal's blood sample; and multiple animal modes may be preset in the methods. In an embodiment, the method further comprises steps of: selecting, in response to a user's instruction of selecting a mode, a corresponding animal mode from the multiple animal modes, wherein each animal mode has a corresponding hemolytic agent and dosage, and a preset temperature range; and then the animal's blood sample is tested according to the selected animal mode. In an embodiment, the animal mode includes one or both of a feline mode and a canine mode, in which the preset temperature range corresponding to the feline mode is 31 degrees Celsius to 40 degrees Celsius, and the preset temperature range corresponding to the canine mode is 28 degrees Celsius to 38 degrees Celsius, preferably, the preset temperature range corresponding to the feline mode and the canine mode is about 35 degrees Celsius.

A few specific examples are described below.

An example of classification and counting of white blood cells of a dog is provided.

First, 1400 uL of a diluent base solution heated to a preset temperature is added to a white blood cell counting chamber.

Next, 9 uL of a blood sample is added to the white blood cell counting chamber, and 700 uL of a diluent is added at the same time to rinse a sampling needle.

Next, the liquid in the white blood cell counting chamber is mixed evenly by means of introducing bubbles at the lower end of the white blood cell counting chamber.

Next, 25 uL of the evenly mixed sample in the white blood cell counting chamber can be aspirated out for the counting in a red blood cell channel.

Next, 0.29 mL of a hemolytic agent is added to the white blood cell counting chamber.

Next, the liquid in the white blood cell counting chamber is mixed evenly by means of introducing bubbles at the lower end of the white blood cell counting chamber. The temperature of the liquid in the white blood cell counting chamber is within a preset range, for example, 28 degrees Celsius to 38 degrees Celsius. At this temperature, red blood cells are lysed by the hemolytic agent, and in white blood cells, under the action of the hemolytic agent, the shrinkage rates of lymphocytes, monocytes and neutrophils are increased, while the shrinkage rates of eosinophils and the basophils are relatively slow. The liquid in the white blood cell counting chamber passes through an orifice in the white blood cell counting chamber under negative pressure, and thus signals from the white blood cell channel are measured to obtain the white blood cell histogram as shown in FIG. 11, so as to complete four-classification and counting or five-classification and counting of white blood cells, for example, finally obtain many test results from the white blood cell channel, such as the count of white blood cells, the count of lymphocytes, the count of monocytes, the count of neutrophils, the count of eosinophils, the count of basophils, the percentage of lymphocytes, the percentage of monocytes, the percentage of neutrophils, the percentage of eosinophils, the percentage of basophils, etc.

Another example of classification and counting of white blood cells of a dog is provided.

First, 1400 uL of a diluent base solution heated to a preset temperature is added to a white blood cell counting chamber.

Next, 9 uL of a blood sample is added to the white blood cell counting chamber, and 700 uL of a diluent is added at the same time to rinse a sampling needle.

Next, the liquid in the white blood cell counting chamber is mixed evenly by means of introducing bubbles at the lower end of the white blood cell counting chamber.

Next, 20 uL of the evenly mixed sample in the white blood cell counting chamber can be aspirated out for the counting in a red blood cell channel.

Next, 0.23 mL of a hemolytic agent is added to the white blood cell counting chamber.

Next, the liquid in the white blood cell counting chamber is mixed evenly by means of introducing bubbles at the lower end of the white blood cell counting chamber. The function of the hemolytic agent is to lyse red blood cells, and under the action of the hemolytic agent, the white blood cells are separated into three populations, including a lymphocyte population, a monocyte population, and a granulocyte population (including neutrophils, eosinophils and basophils), and the liquid in the white blood cell counting chamber passes through an orifice of the white blood cell counting chamber under negative pressure. Therefore, a first measurement is performed on signals from the white blood cell channel to obtain the white blood cell histogram as shown in FIG. 12A, and thus obtain the classification value of lymphocytes (the percentage of lymphocytes), the classification value of monocytes (the percentage of monocytes) and the classification value of granulocytes (the percentage of granulocytes).

Next, 0.26 mL of a hemolytic agent is added to the white blood cell counting chamber again.

Next, the liquid in the white blood cell counting chamber is mixed evenly by means of introducing bubbles at the lower end of the white blood cell counting chamber.

The second addition of the hemolytic agent is to increase the shrinkage rates of lymphocytes, monocytes and neutrophils, while the shrinkage rates of eosinophils and basophils are relatively slow. The eosinophils and the basophils are at the rightmost of the white blood cell histogram. The liquid in the white blood cell counting chamber passes through the orifice of the white blood cell counting chamber under negative pressure to perform a second measurement on signals from the white blood cell channel to obtain the white blood cell histogram as shown in FIG. 12B, thereby obtaining the count of white blood cells, the percentage of eosinophils, the count of eosinophils, the percentage of basophils and the count of basophils.

Finally, other parameters are calculated according to the results of the two histograms as follows:

the count of lymphocytes=the count of white blood cells*the percentage of lymphocytes;

the count of monocytes=the count of white blood cells*the percentage of monocytes;

the percentage of neutrophils=the percentage of granulocytes−the percentage of eosinophils−the percentage of basophils; and

the count of neutrophils=the count of white blood cells*the percentage of neutrophils;

Through the counting of the first and second histograms, it is possible to obtain multiple test results from the white blood cell channel, such as the count of white blood cells, the count of lymphocytes, the count of monocytes, the count of neutrophils, the count of eosinophils, the count of basophils, the percentage of lymphocytes, the percentage of monocytes, the percentage of neutrophils, the percentage of eosinophils, the percentage of basophils, etc.

Another example of classification and counting of white blood cells of a dog is provided.

First, 1400 uL of a diluent base solution heated to a preset temperature is added to a white blood cell counting chamber.

Next, 9 uL of a blood sample is added to the white blood cell counting chamber, and 700 uL of a diluent is added at the same time to rinse a sampling needle.

Next, the liquid in the white blood cell counting chamber is mixed evenly by means of introducing bubbles at the lower end of the white blood cell counting chamber.

Next, 20 uL of the evenly mixed sample in the white blood cell counting chamber can be aspirated out for the counting in a red blood cell channel.

Next, 0.3 mL of a hemolytic agent is added to the white blood cell counting chamber.

Next, the liquid in the white blood cell counting chamber is mixed evenly by means of introducing bubbles at the lower end of the white blood cell counting chamber. The function of the hemolytic agent is to lyse red blood cells, and under the action of the hemolytic agent, the white blood cells are separated into three populations, including a lymphocyte population, a monocyte population, and a granulocyte population (including neutrophils, eosinophils and basophils), and the liquid in the white blood cell counting chamber passes through an orifice of the white blood cell counting chamber under negative pressure. Therefore, a first measurement is performed on signals from the white blood cell channel to obtain the white blood cell histogram as shown in FIG. 12A, and thus obtain the classification value of lymphocytes (the percentage of lymphocytes), the classification value of monocytes (the percentage of monocytes) and the classification value of granulocytes (the percentage of granulocytes).

Next, waiting for 10 to 20 seconds, the hemolytic agent further acts on white blood cells and red blood cells.

Next, the liquid in the white blood cell counting chamber is mixed evenly by means of introducing bubbles at the lower end of the white blood cell counting chamber.

The function of waiting for 10 to 20 seconds is on the one hand to allow the hemolytic agent to further act on red blood cells such that there are no red blood cell fragments in the sample to influence the counting of white blood cells, and on the other hand to allow the hemolytic agent to further act on white blood cells such that the shrinkage rates of lymphocytes, monocytes and neutrophils are increased, while the shrinkage rates of eosinophils and basophils are relatively slow. The eosinophils and the basophils are at the rightmost of the white blood cell histogram. The liquid in the white blood cell counting chamber passes through the orifice of the white blood cell counting chamber under negative pressure to perform a second measurement on signals from the white blood cell channel to obtain the white blood cell histogram as shown in FIG. 12B, thereby obtaining the count of white blood cells, the percentage of eosinophils, the count of eosinophils, the percentage of basophils and the count of basophils.

Finally, other parameters are calculated according to the results of the two histograms, and the calculation process is similar to the calculation in the example of the dog above, which will not be repeated here.

An example of classification and counting of white blood cells of a cat is provided.

First, 1400 uL of a diluent base solution heated to a preset temperature is added to a white blood cell counting chamber.

Next, 9 uL of a blood sample is added to the white blood cell counting chamber, and 700 uL of a diluent is added at the same time to rinse a sampling needle.

Next, the liquid in the white blood cell counting chamber is mixed evenly by means of introducing bubbles at the lower end of the white blood cell counting chamber.

Next, 25 uL of the evenly mixed sample in the white blood cell counting chamber can be aspirated out for the counting in a red blood cell channel.

Next, 0.29 mL of a hemolytic agent is added to the white blood cell counting chamber.

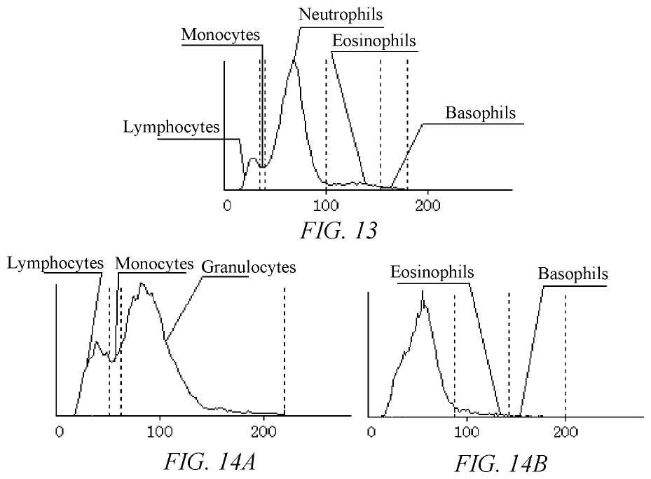

Next, the liquid in the white blood cell counting chamber is mixed evenly by means of introducing bubbles at the lower end of the white blood cell counting chamber. The temperature of the liquid in the white blood cell counting chamber is within a preset range, for example, 28 degrees Celsius to 38 degrees Celsius. At this temperature, red blood cells are lysed by the hemolytic agent, and in white blood cells, under the action of the hemolytic agent, the shrinkage rates of lymphocytes, monocytes and neutrophils are increased, while the shrinkage rates of eosinophils and the basophils are relatively slow. The liquid in the white blood cell counting chamber passes through an orifice in the white blood cell counting chamber under negative pressure, and thus signals from the white blood cell channel are measured to obtain the white blood cell histogram as shown in FIG. 13, so as to complete four-classification and counting or five-classification and counting of white blood cells, for example, finally obtain many test results from the white blood cell channel, such as the count of white blood cells, the count of lymphocytes, the count of monocytes, the count of neutrophils, the count of eosinophils, the count of basophils, the percentage of lymphocytes, the percentage of monocytes, the percentage of neutrophils, the percentage of eosinophils, the percentage of basophils, etc.

An example of classification and counting of white blood cells of a cat is provided.

First, 1400 uL of a diluent base solution heated to a preset temperature is added to a white blood cell counting chamber.

Next, 9 uL of a blood sample is added to the white blood cell counting chamber, and 700 uL of a diluent is added at the same time to rinse a sampling needle.

Next, the liquid in the white blood cell counting chamber is mixed evenly by means of introducing bubbles at the lower end of the white blood cell counting chamber.

Next, 20 uL of the evenly mixed sample in the white blood cell counting chamber can be aspirated out for the counting in a red blood cell channel.

Next, 0.26 mL of a hemolytic agent is added to the white blood cell counting chamber.

Next, the liquid in the white blood cell counting chamber is mixed evenly by means of introducing bubbles at the lower end of the white blood cell counting chamber. The function of the hemolytic agent is to lyse red blood cells, and under the action of the hemolytic agent, the white blood cells are separated into three populations, including a lymphocyte population, a monocyte population, and a granulocyte population (including neutrophils, eosinophils and basophils), and the liquid in the white blood cell counting chamber passes through an orifice of the white blood cell counting chamber under negative pressure. Therefore, a first measurement is performed on signals from the white blood cell channel to obtain the white blood cell histogram as shown in FIG. 14A, and thus obtain the classification value of lymphocytes (the percentage of lymphocytes), the classification value of monocytes (the percentage of monocytes) and the classification value of granulocytes (the percentage of granulocytes).

Next, 0.23 mL of a hemolytic agent is added to the white blood cell counting chamber again.

Next, the liquid in the white blood cell counting chamber is mixed evenly by means of introducing bubbles at the lower end of the white blood cell counting chamber.

The second addition of the hemolytic agent is to increase the shrinkage rates of lymphocytes, monocytes and neutrophils, while the shrinkage rates of eosinophils and basophils are relatively slow. The eosinophils and the basophils are at the rightmost of the white blood cell histogram. The liquid in the white blood cell counting chamber passes through the orifice of the white blood cell counting chamber under negative pressure to perform a second measurement on signals from the white blood cell channel to obtain the white blood cell histogram as shown in FIG. 14B, thereby obtaining the count of white blood cells, the percentage of eosinophils, the count of eosinophils, the percentage of basophils and the count of basophils.

Finally, other parameters are calculated according to the results of the two histograms, and the calculation process is similar to the calculation in the example of the dog above, which will not be repeated here.

Another example of classification and counting of white blood cells of a cat is provided.

First, 1400 uL of a diluent base solution heated to a preset temperature is added to a white blood cell counting chamber.

Next, 9 uL of a blood sample is added to the white blood cell counting chamber, and 700 uL of a diluent is added at the same time to rinse a sampling needle.

Next, the liquid in the white blood cell counting chamber is mixed evenly by means of introducing bubbles at the lower end of the white blood cell counting chamber.

Next, 20 uL of the evenly mixed sample in the white blood cell counting chamber can be aspirated out for the counting in a red blood cell channel.

Next, 0.26 mL of a hemolytic agent is added to the white blood cell counting chamber.

Next, the liquid in the white blood cell counting chamber is mixed evenly by means of introducing bubbles at the lower end of the white blood cell counting chamber. The function of the hemolytic agent is to lyse red blood cells, and under the action of the hemolytic agent, the white blood cells are separated into three populations, including a lymphocyte population, a monocyte population, and a granulocyte population (including neutrophils, eosinophils and basophils), and the liquid in the white blood cell counting chamber passes through an orifice of the white blood cell counting chamber under negative pressure. Therefore, a first measurement is performed on signals from the white blood cell channel to obtain the white blood cell histogram as shown in FIG. 14A, and thus obtain the classification value of lymphocytes (the percentage of lymphocytes), the classification value of monocytes (the percentage of monocytes) and the classification value of granulocytes (the percentage of granulocytes).

Next, waiting for 12 seconds, the hemolytic agent further acts on white blood cells and red blood cells.

Next, the liquid in the white blood cell counting chamber is mixed evenly by means of introducing bubbles at the lower end of the white blood cell counting chamber.

The function of waiting for 12 seconds is on the one hand to allow the hemolytic agent to further act on red blood cells such that there are no red blood cell fragments in the sample to influence the counting of white blood cells, and on the other hand to allow the hemolytic agent to further act on white blood cells such that the shrinkage rates of lymphocytes, monocytes and neutrophils are increased, while the shrinkage rates of eosinophils and basophils are relatively slow. The eosinophils and the basophils are at the rightmost of the white blood cell histogram. The liquid in the white blood cell counting chamber passes through the orifice of the white blood cell counting chamber under negative pressure to perform a second measurement on signals from the white blood cell channel to obtain the white blood cell histogram as shown in FIG. 14B, thereby obtaining the count of white blood cells, the percentage of eosinophils, the count of eosinophils, the percentage of basophils and the count of basophils.

Finally, other parameters are calculated according to the results of the two histograms, and the calculation process is similar to the calculation in the example of the dog above, which will not be repeated here.

The description has been made with reference to various exemplary embodiments herein. However, those skilled in the art would have appreciated that changes and modifications could have been made to the exemplary embodiments without departing from the scope herein. For example, various operation steps and assemblies for executing operation steps may be implemented in different ways according to a specific application or considering any number of cost functions associated with the operation of the system (for example, one or more steps may be deleted, modified or incorporated into other steps).

In the above embodiments, the disclosure may be implemented in whole or in part by software, hardware, firmware, or any combination thereof. In addition, as understood by those skilled in the art, the principles herein may be reflected in a computer program product on a computer-readable storage medium that is pre-installed with computer-readable program codes. Any tangible, non-transitory computer-readable storage medium can be used, including magnetic storage devices (hard disks, floppy disks, etc.), optical storage devices (CD-ROM, DVD, Blu Ray disks, etc.), flash memories, and/or the like. These computer program instructions can be loaded onto a general-purpose computer, a dedicated computer, or other programmable data processing device to form a machine, such that these instructions executed on a computer or other programmable data processing apparatus can generate an apparatus that implements a specified function. These computer program instructions can also be stored in a computer-readable memory that can instruct a computer or other programmable data processing device to operate in a specific manner, such that the instructions stored in the computer-readable memory can form a manufactured product, including an implementation apparatus that implements a specified function. The computer program instructions can also be loaded onto a computer or other programmable data processing device, such that a series of operating steps are executed on the computer or other programmable device to produce a computer-implemented process, such that the instructions executed on the computer or other programmable device can provide steps for implementing a specified function.

Although the principles herein have been shown in various embodiments, many modifications of structures, arrangements, ratios, elements, materials, and components that are particularly suitable for specific environments and operating requirements can be made without departing from the principles and scope of the disclosure. The above modifications and other changes or amendments will be included within the scope herein.

The above specific description has been described with reference to various embodiments. However, those skilled in the art would have appreciated that various modifications and changes could have been made without departing from the scope of the disclosure. Therefore, consideration of the disclosure will be in an illustrative rather than a restrictive sense, and all such modifications will be included within the scope thereof. Likewise, the advantages of various embodiments, other advantages, and the solutions to problems have been described above. However, the benefits, advantages, solutions to problems, and any elements that can produce these, or solutions that make them more explicit, should not be interpreted as critical, necessary, or essential. The term “comprising” and any other variants thereof used herein are non-exclusive, such that the process, method, document, or device that includes a list of elements includes not only these elements, but also other elements that are not explicitly listed or do not belong to the process, method, system, document, or device. Furthermore, the term “coupling” and any other variations thereof used herein refer to physical connection, electrical connection, magnetic connection, optical connection, communication connection, functional connection, and/or any other connection.

Those skilled in the art will recognize that many changes can be made to the details of the above-described embodiments without departing from the basic principles of the disclosure. Therefore, the scope of the disclosure should be determined only by the claims as follows.

1. A method for classifying white blood cells based on an impedance method, comprising:
adding a diluent to a white blood cell counting chamber;
adding a sample to be analyzed to the white blood cell counting chamber;
adding a hemolytic agent to the white blood cell counting chamber at least once;
controlling a temperature of the liquid in the white blood cell counting chamber to be within a preset temperature range; and
testing the liquid in the white blood cell counting chamber to perform at least four-classification and counting of white blood cells.

2. The method of claim 1, wherein the step of controlling a temperature of the liquid in the white blood cell counting chamber to be within a preset temperature range comprises: heating the diluent to a certain temperature, and then adding the diluent to the white blood cell counting chamber so as to control the temperature of the liquid in the white blood cell counting chamber to be within the preset temperature range; or
wherein the step of controlling a temperature of the liquid in the white blood cell counting chamber to be within a preset temperature range comprises: heating the liquid in the white blood cell counting chamber so as to control the temperature of the liquid in the white blood cell counting chamber to be within the preset temperature range.

3. (canceled)

4. The method of claim 1, comprising:
adding the hemolytic agent to the white blood cell counting chamber only once such that an amount of red blood cell fragments in the sample is less than a preset threshold;
testing the liquid in the white blood cell counting chamber once to obtain a white blood cell histogram; and
performing the at least four-classification and counting of white blood cells according to the white blood cell histogram from said testing.

5. The method of claim 1, comprising:
adding a first hemolytic agent to the white blood cell counting chamber;
testing the liquid in the white blood cell counting chamber once to obtain a first white blood cell histogram;
adding a second hemolytic agent to the white blood cell counting chamber such that an amount of red blood cell fragments in the sample is less than a preset threshold;
testing the liquid in the white blood cell counting chamber once to obtain a second white blood cell histogram; and
performing the at least four-classification and counting of white blood cells according to the first white blood cell histogram and the second white blood cell histogram; or
wherein the method comprises:
adding the hemolytic agent to the white blood cell counting chamber only once;
testing the liquid in the white blood cell counting chamber once to obtain a first white blood cell histogram;
waiting for a preset period of time, so that the hemolytic agent continues to act on the sample such that an amount of red blood cell fragments in the sample is less than a preset threshold;
testing the liquid in the white blood cell counting chamber once to obtain a second white blood cell histogram; and
performing the at least four-classification and counting of white blood cells according to the first white blood cell histogram and the second white blood cell histogram.

6-7. (canceled)

8. The method of claim 5, comprising:
obtaining a percentage of lymphocytes, a percentage of monocytes and a percentage of granulocytes according to the first white blood cell histogram;
obtaining a count of white blood cells, a percentage of eosinophils and a count of eosinophils according to the second white blood cell histogram;
subtracting the percentage of eosinophils from the percentage of granulocytes to obtain a percentage of neutrophils; and
calculating a count of lymphocytes, a count of monocytes and a count of neutrophils according to the count of white blood cells, the percentage of lymphocytes, the percentage of monocytes and the percentage of neutrophils; or
wherein the method comprises:
obtaining a percentage of lymphocytes, a percentage of monocytes and a percentage of granulocytes according to the first white blood cell histogram;
obtaining a count of white blood cells, a percentage of basophils and a count of basophils according to the second white blood cell histogram;
subtracting the percentage of basophils from the percentage of granulocytes to obtain a percentage of a total of neutrophils and eosinophils; and
calculating a count of lymphocytes, a count of monocytes, and a total count of neutrophils and eosinophils according to the count of white blood cells, the percentage of lymphocytes, the percentage of monocytes, and the percentage of the total of neutrophils and eosinophils; or
wherein the method comprises:
obtaining a percentage of lymphocytes, a percentage of monocytes and a percentage of granulocytes according to the first white blood cell histogram;
obtaining a count of white blood cells, a percentage of basophils, a count of basophils, a percentage of eosinophils and a count of eosinophils according to the second white blood cell histogram;
subtracting the percentage of basophils and the percentage of eosinophils from the percentage of granulocytes to obtain a percentage of neutrophils; and
calculating a count of lymphocytes, a count of monocytes and a count of neutrophils according to the count of white blood cells, the percentage of lymphocytes, the percentage of monocytes and the percentage of neutrophils.

9-10. (canceled)

11. The method of claim 8, comprising: processing the first white blood cell histogram to remove influence of the red blood cell fragments, and obtaining the percentage of lymphocytes, the percentage of monocytes and the percentage of granulocytes according to the first white blood cell histogram after the removal of influence of the red blood cell fragments.

12. The method of claim 1, wherein the sample is a blood sample from an animal.

13. The method of claim 12, further comprising:
selecting, in response to a user's instruction of selecting a mode, a corresponding animal mode from multiple animal modes, wherein each animal mode has a corresponding hemolytic agent and dosage, and a preset temperature range; and
testing the animal's blood sample according to the selected animal mode.

14. The method of claim 13, wherein the animal modes include one or both of a feline mode and a canine mode, wherein the preset temperature range corresponding to the feline mode is 31 degrees Celsius to 40 degrees Celsius, and the preset temperature range corresponding to the canine mode is 28 degrees Celsius to 38 degrees Celsius.

15. The method of claim 14, wherein the preset temperature range corresponding to the feline mode and the canine mode is about 35 degrees Celsius.

16. A method for classifying white blood cells based on an impedance method, comprising:
adding a diluent to a white blood cell counting chamber;
aspirating a sample to be analyzed by a sampling needle assembly, and
discharging a portion of the sample to the white blood cell counting chamber;
adding a first hemolytic agent to the white blood cell counting chamber;
testing the liquid in the white blood cell counting chamber once to obtain a first white blood cell histogram;
draining and cleaning the white blood cell counting chamber;
adding a diluent to the white blood cell counting chamber;
discharging at least a portion of the remaining sample to the white blood cell counting chamber by the sampling needle assembly;
adding a second hemolytic agent to the white blood cell counting chamber;
testing the liquid in the white blood cell counting chamber once to obtain a second white blood cell histogram; and
performing at least four-classification and counting of white blood cells according to the first white blood cell histogram and the second white blood cell histogram, wherein an amount of red blood cell fragments in the second white blood cell histogram is less than a preset threshold; and
wherein a temperature of the liquid in the white blood cell counting chamber is controlled to be within a preset temperature range before each testing.

17. The method of claim 16, wherein the first hemolytic agent and the second hemolytic agent are a same hemolytic agent.

18. The method of claim 16, wherein a dosage of the first hemolytic agent is less than a dosage of the second hemolytic agent.

19. The method of claim 16, comprising: obtaining a percentage of lymphocytes, a percentage of monocytes and a percentage of granulocytes according to the first white blood cell histogram; and
obtaining a count of white blood cells, a percentage of eosinophils and a count of eosinophils according to the second white blood cell histogram; or obtaining a count of white blood cells, a percentage of basophils and a count of basophils according to the second white blood cell histogram; or obtaining a count of white blood cells, a percentage of basophils, a count of basophils, a percentage of eosinophils and a count of eosinophils according to the second white blood cell histogram.

20. The method of claim 16, wherein the step of controlling a temperature of the liquid in the white blood cell counting chamber to be within a preset temperature range comprises: heating the diluent to a certain temperature, and then adding the diluent to the white blood cell counting chamber, such that the temperature of the liquid in the white blood cell counting chamber is controlled to be within the preset temperature range; or heating the liquid in the white blood cell counting chamber, such that the temperature of the liquid in the white blood cell counting chamber is controlled to be within the preset temperature range.

21. A cell analyzer, comprising:
a white blood cell counting chamber comprising an orifice;
a sampling needle assembly for discharging a sample to be analyzed into the white blood cell counting chamber;
a diluent delivery component for delivering a diluent to the white blood cell counting chamber;
a hemolytic agent delivery component for delivering a hemolytic agent to the white blood cell counting chamber;
a pressure source component for providing pressure to enable the liquid in the white blood cell counting chamber to pass through the orifice;
a resistive detector for testing the liquid passing through the orifice;
a heating component for controlling a temperature of the liquid in the white blood cell counting chamber; and
a controller and a processor, wherein
the controller controls the diluent delivery component to deliver the diluent to the white blood cell counting chamber;
the controller controls the sampling needle assembly to add the sample to be analyzed to the white blood cell counting chamber;
the controller controls the hemolytic agent delivery component to add the hemolytic agent to the white blood cell counting chamber at least once;
the controller controls the heating component to control the temperature of the liquid in the white blood cell counting chamber to be within a preset temperature range;
the controller controls the pressure source component to provide pressure to enable the liquid in the white blood cell counting chamber to pass through the orifice, and controls the resistive detector to test the liquid passing through the orifice; and
the processor performs at least four-classification and counting of white blood cells according to data output by the resistive detector.

22. The cell analyzer of claim 21, wherein the diluent delivery component comprises a helical pipeline in fluid communication with the white blood cell counting chamber, and the helical pipeline is provided with the heating component; and the controller controls the heating component to heat the diluent in the helical pipeline that flows to the white blood cell counting chamber, such that the temperature of the liquid in the white blood cell counting chamber is controlled to be within the preset temperature range.

23. The cell analyzer of claim 21, wherein the heating component comprises a container having a liquid inlet and a liquid outlet, and a heating member for heating a liquid in the container and a temperature sensor for detecting a temperature of the liquid in the container, the heating member and the temperature sensor being arranged in the container, wherein the liquid inlet is in liquid communication with the diluent delivery component through a pipeline, the liquid outlet is connected to the white blood cell counting chamber through a pipeline, the diluent delivered by the diluent delivery component enters the container via the liquid inlet and flows out of the container via the liquid outlet and then enters the white blood cell counting chamber, and the controller controls the heating member for heating when it is determined that the temperature of the diluent in the container is lower than a first temperature according to the data of the temperature sensor, and controls the heating member to stop heating when it is determined that the temperature of the diluent in the container is higher than a second temperature.

24. The cell analyzer of claim 21, further comprising a temperature sensor provided in the white blood cell counting chamber for detecting a temperature of the liquid in the white blood cell counting chamber, wherein the heating component is arranged on the white blood cell counting chamber for heating the liquid in the white blood cell counting chamber; and the controller controls the heating component for heating when it is determined that the temperature of the liquid in the white blood cell counting chamber is lower than the preset temperature range according to the data of the temperature sensor, and controls the heating component to stop heating when it is determined that the temperature of the liquid in the white blood cell counting chamber is higher than the preset temperature range.

25. The cell analyzer of claim 21, wherein
the controller controls the hemolytic agent delivery component to add the hemolytic agent to the white blood cell counting chamber only once, such that an amount of red blood cell fragments in the sample is less than a preset threshold;
the controller controls the resistive detector to perform one testing on the liquid in the white blood cell counting chamber; and
the processor obtains a white blood cell histogram according to the data output by the resistive detector, and performs the at least four-classification and counting of white blood cells according to the white blood cell histogram; or
wherein
the controller controls the hemolytic agent delivery component to add a first hemolytic agent to the white cell counting chamber;
the controller controls the resistive detector to perform one testing on the liquid in the white blood cell counting chamber, and the processor obtains a first white blood cell histogram according to the data output by the resistive detector in said testing;
the controller controls the hemolytic agent delivery component to add a second hemolytic agent to the white cell counting chamber;
the controller controls the resistive detector to perform one testing on the liquid in the white blood cell counting chamber, and the processor obtains a second white blood cell histogram according to the data output by the resistive detector in said testing, wherein an amount of red blood cell fragments in the second white blood cell histogram is less than a preset threshold; and
the processor performs the at least four-classification and counting of white blood cells according to the first white blood cell histogram and the second white blood cell histogram; or
wherein
the controller controls the hemolytic agent delivery component to add the hemolytic agent to the white blood cell counting chamber only once;
the controller controls the resistive detector to perform one testing on the liquid in the white blood cell counting chamber, and the processor obtains a first white blood cell histogram according to the data output by the resistive detector in said testing;
the controller controls, after waiting for a preset period of time, the resistive detector to perform one testing on the liquid in the white blood cell counting chamber, and the processor obtains a second white blood cell histogram according to the data output by the resistive detector in said testing, wherein an amount of red blood cell fragments in the second white blood cell histogram is less than a preset threshold; and
the processor performs the at least four-classification and counting of white blood cells according to the first white blood cell histogram and the second white blood cell histogram.

26-31. (canceled)
